(19) United States
(12) Patent Application Publication
(10) Pub. No.: US 2019/0073452 A1
(43) Pub. Date: Mar. 7, 2019

(54) METHOD FOR DETERMINING THE IN VIVO COMPARABILITY OF A BIOLOGIC DRUG AND A REFERENCE DRUG

(71) Applicant: BIOANALYTIX, Inc., Cambridge, MA (US)

(72) Inventors: Lily Y.T. Li, Lexington, MA (US); Kirtland G. Poss, Marblehead, MA (US); Shiaw-Lin Wu, Weston, MA (US); Robert Garnick, Las Vegas, NV (US)

(73) Assignee: BIOANALYTIX, Inc., Cambridge, MA (US)

(21) Appl. No.: 15/762,121

(22) PCT Filed: Sep. 23, 2016
(86) PCT No.: PCT/US2016/053434

§ 371 Date: Mar. 22, 2018

Related U.S. Application Data
(60) Provisional application No. 62/232,894, filed on Sep. 25, 2015.

(51) Int. Cl. G06F 19/00; G01N 33/68; G06F 19/26

(52) U.S. Cl. CPC G06F 19/708; G06F 19/26; G01N 33/6851; G01N 33/6848

(57) ABSTRACT

The invention provides a mass-spectroscopic approach for assessing and determining the in vivo comparability of a candidate biologic molecule to a reference biologic molecule. The results can be presented in the form of an in vivo comparability profile, which can serve as a development tool, e.g., as a guide or target for the development of biologies, biosimilars, or gene therapy-based drugs. The invention further provides an approach using an in vivo comparability profile for measuring the similarity of two biologies, for example, a biosimilar and a reference approved biologic or manufacturing lots of the same biologic to confirm acceptable release criteria for a particular manufacturing lot.

### CROSS-REFERENCE TO RELATED APPLICATIONS

This application claims the benefit and priority to U.S. Application No. 62/232,894, filed Sep. 25, 2015, the entire contents of which are incorporated herein by reference.

### FIELD OF THE INVENTION

The invention relates generally to a method of analyzing and determining the in vivo comparability of a candidate biologic drug to a reference biologic drug when administered to a subject.

### BACKGROUND

The development of biologic drug products over the years has often been hindered by an inability to effectively characterize the structural features of the biologic drug, in particular the structural features of a biologic drug that are actually affected by in vivo residence within a subject. This limitation becomes particularly significant when attempting to assess the similarity of a generic biologic (for example, a biosimilar) or a gene therapy produced protein that is intended to replicate or mimic the target protein of interest.

Biologics are highly complex molecules that often are modified in vivo upon administration to a subject. For example, biologics may be subjected to degradation or other chemical changes by exposure to proteases and other enzymes in vivo. Similarly, carbohydrates present, for example, in glycoproteins or glycolipids may be modified in vivo, for example, via deglycosylation at differing N-linked or O-linked sites and by different amounts of deglycosylation. Proper disulfide bonding within a molecule and/or between molecules typically is critical for efficacy, and oxidation of the disulfide bonds may lead to inoperative misfolded proteins, or may otherwise affect the structure, function and/or stability of the resulting molecule. Similarly the biologics may be deaminated, oxidized, methylated or otherwise derivatized in vivo, and the modifications may affect the structure, function and/or stability of the resulting molecule. While metabolism of the biologic may be critical for its normal function, improper metabolism may decrease the drug's efficacy, or create unknown and unpredictable off-target effects.

The established approaches for producing small molecule drugs, involving precise chemical replication and demonstration of biological equivalence, is clearly scientifically insufficient for far more complex biological products. As a result, there is still an ongoing need for a process for definitively characterizing and comparing the key structures of a candidate biologic drug and for a method of determining how the candidate biologic drug is processed in vivo relative to a reference biologic.

### SUMMARY

The inventors have developed an approach to enable developers, manufacturers and potentially regulators of biologic drugs (for example, biologics, biosimilars, or gene therapy-based drugs) to better understand how a candidate biologic drug behaves in vivo in a subject relative to a reference biologic drug, and therefore to be able to determine the in vivo comparability profile of one biologic drug or drug candidate versus another. The approach involves the creation of an in vivo comparability profile (or map, statistical analysis by attribute, or fingerprint analysis) that comprises a set of structural features of a candidate molecule and metabolites following administration of the candidate molecule to a subject that demonstrate the in vivo similarity of the candidate molecule when compared to a reference molecule. The profile can then be used to compare the behavior of two biologic molecules (for example, a candidate biologic versus a native molecule, or a candidate biosimilar versus an approved reference molecule) to show how the two molecules structurally change or progress in vivo upon and following administration to a subject. Accordingly, the invention provides a systematic, sensitive and efficient method for determining how a molecule of interest behaves, progresses or changes structurally in vivo relative to a reference molecule.

In one aspect, the invention provides a method of measuring in vivo comparability of a candidate biologic drug to a reference biologic drug. The method comprises the steps of: (a) generating, through mass spectroscopic analysis, data indicative of the structure of the candidate biologic drug or a metabolite thereof following extraction from a sample, for example, a cell, tissue or body fluid sample, removed from a subject, for example, a human or animal, at a first time interval following administration of the candidate biologic drug to the subject; and (b) comparing the data generated during step (a) to mass spectroscopic analysis data indicative of the structure of the reference biologic drug or a metabolite thereof generated through analysis of a sample taken at the same time interval from a subject to whom the reference biologic drug had been administered, thereby to produce an in vivo comparability profile of the candidate biologic drug relative to the reference biologic drug.

It is understood that the initial step (step (a)) can be repeated at two or more time intervals following administration so as to produce a comparability profile of the candidate biologic drug relative to the reference biologic drug as a function of time in vivo.

The method may further comprise the additional step of using the in vivo comparability profile to determine the metabolic or structural comparability of the candidate biologic drug to the reference biologic drug. The in vivo comparability profile can contain data indicative of the structural or metabolic profile of the reference biologic drug and the structural or metabolic profile of the candidate biologic drug over time following administration to the subject.

The in vivo comparability profile comprises one or more sets of data indicative of (a) a structural feature that is the same or different when comparing the candidate biologic drug or a metabolite thereof and the reference biologic drug or a corresponding metabolite thereof; and optionally, when available, (b) data indicative of whether differences in a structural feature between the candidate biologic drug and the reference biologic drug substantially affect a biological activity of the candidate biologic drug. The resulting data can be used to create a record of structural features of the candidate molecule and/or reference molecule, for example, a profile, which identifies and characterizes parts of the molecular structure that are modified during in vivo residence. The profile can reveal which particular attributes or modifications affect the molecule's stability and/or activity, and which attributes or modifications do not materially affect stability and/or activity. The profile can provide evidence of how the biologic drug (the candidate drug, the reference drug, or both the candidate drug and the reference drug) is processed within the subject, and thus can serve as a comparison of how the candidate molecule (for example, biosimilar) and the reference molecule (for example, the approved biologic) behave in vivo following administration to a subject.

The resulting in vivo comparability profile may comprise sufficient detail to permit qualification of the candidate biologic drug as (i) biosimilar to the reference biologic drug, (ii) substantially the same as the reference biologic drug, or (iii) interchangeable with the reference biologic drug. Where the candidate biologic drug and the reference biologic correspond to different manufacturing lots of the same biologic drug, the comparability profile generated in step (b) may comprise sufficient detail to serve as criteria to qualify the release of the manufacturing lot containing the candidate biologic drug.

It is contemplated, that the subject in step (a) is a human and/or the subject in step (b) is a human. Optionally, the subject in step (a) can be different from the subject in step (b) (i.e., a different person) or the subject in step (a) can be the same as the subject in step (b) (i.e., the same person). Similarly, it is contemplated, that the subject in step (a) is an animal and/or the subject in step (b) is an animal. Optionally, the subject in step (a) can be different from the subject in step (b) (i.e., a different animal) or the subject in step (a) can be the same as the subject in step (b) (i.e., the same animal). it is contemplated that the foregoing method can be used to determine the in vivo comparability of any candidate biologic drug, including, without limitation, a protein, peptide, glycoprotein lipid, or glycolipid. For example, in the case of a protein, the in viva behavioral characteristics of the candidate protein, for example, the candidate therapeutic protein can be compared to a reference, for example, the native protein, or an approved reference protein in the case of a biosimilar. Similarly, it is contemplated that, when the candidate biologic drug is an expression product produced by expression of a gene during gene therapy (for example, protein or mRNA) the reference biologic drug can be the native expression product (for example, the respective endogenous protein or mRNA) whose level is intended to be augmented or modulated by the gene therapy.

Further, the in vivo comparability profile can be used to facilitate the production of innovator biologics. For example, a candidate biologic drug can be developed using various different raw materials, expression conditions, purification techniques and reagents until a biologic product is produced where the resulting in vivo comparability profile shows that the biologic drug being developed has similar structural features (and optional functional attributes) to the endogenous reference protein. Furthermore, the in vivo comparability profile can be used to facilitate the development of a biosimilar. For example, a biosimilar can be developed using various expression constructs, expression conditions, purification and formulation techniques until a biosimilar is produced where the resulting in vivo comparability profile shows that the biosimilar actually has similar structural features (and optional functional attributes) as the approved reference biologic in vivo. Furthermore, the in vivo comparability profile can be used to facilitate the development of gene therapy-based approaches to replace or augment an endogenous protein. For example, an expression construct can be developed, modified, as appropriate, and tested to produce an expression product (for example, protein) where the resulting in vivo comparability profile shows that the expression product has the relevant or similar structural features (and optional functional attributes) as the endogenous molecule (for example, endogenous protein) that is intended to be augmented or modulated by the gene therapy.

In another aspect, the invention provides a method of measuring in vivo comparability of a candidate biologic drug to a reference biologic drug, wherein the candidate biologic drug is a protein produced by expression of a gene via gene therapy that corresponds to a native protein whose level is intended to be augmented or modulated by the gene therapy. In such a situation, the reference biologic drug can be a recombinant protein produced in a cell-line that corresponds to the native protein. The method comprises the steps of: (a) generating through mass spectroscopic analysis data indicative of the structure of the candidate biologic drug or a metabolite thereof following extraction from a sample (for example, a cell, tissue or body fluid sample) removed from a subject (for example, a human or animal) at a first time interval following administration of the candidate biologic drug to the subject; and (b) comparing the data generated during step (a) to mass spectroscopic analysis data indicative of the structure of the reference biologic drug, thereby to produce an in vivo comparability profile of the candidate biologic drug to the reference biologic drug.

The method can further comprise repeating step (a) at two or more time intervals following administration so as to produce a comparability profile of the candidate biologic drug relative to the reference biologic drug as a function of time in vivo.

The resulting in vivo comparability profile can be of sufficient detail to permit qualification of the candidate biologic drug as substantially the same as the reference biologic drug. The in vivo comparability profile comprises one or more sets of data indicative of (a) a structural feature that is the same or different when comparing the candidate biologic drug or a metabolite thereof and the reference biologic drug or a corresponding metabolite thereof; and optionally, when available, (b) data indicative of whether differences in a structural feature between the candidate biologic drug and the reference biologic drug substantially affect a biological activity of the candidate biologic drug.

In each of the foregoing aspects, the mass spectrometry analysis data can be generated by fragmenting or chemically modifying, as appropriate, the reference biologic drug, the candidate biologic drug, or metabolites thereof before or while subjecting the sample to mass spectrometric analysis. The generation of mass spectrometry data can be obtained using one or more techniques selected from the group consisting of electron transfer dissociation mass spectrometry, collision induced dissociation mass spectrometry, higher-energy collisional dissociation mass spectrometry, electron capture dissociation mass spectrometry, infrared multi-photon dissociation mass spectrometry, ultraviolet photodissociation mass spectrometry, hydrogen/deuterium exchange mass spectrometry, MS², MS³, and LC-MS. Tandem mass spectrometry data can be acquired using techniques selected from the group consisting of data-dependent acquisition, data-independent acquisition, selected reaction monitoring, and parallel reaction monitoring. In addition, the mass spectrometry analysis data can be generated by one or more analytical techniques selected from the group consisting of fluorescent spectra, light scattering spectroscopy, electrophoresis, selective proteolysis, UV spectra analysis, IR spectra analysis, Hydrogen-Deuterium exchange analysis, and MRI spectra analysis.

In addition, the data indicative of the structure of the candidate molecule or the metabolite thereof comprises a normalized measure of a modified form of a structural attribute corresponding to the amount of a modified form of a structural attribute relative to the total amount of the structural attribute (a sum of modified attribute and unmodified attribute) in the sample being test.

### BRIEF DESCRIPTION OF THE DRAWINGS

FIG. 1 is a flow chart showing different approaches, namely, bottom-up (Fragmentation¹: produced by enzymatic digestion using multiple enzymes), middle down (Fragmentation²: produced by enzymatic digestion using a single enzyme or chemical), or top-down approaches (Fragmentation³: produced via gas phase cleavage using collision induced dissociation (CID) and electron transfer dissociation (ETD)), for characterizing the primary sequence of a biologic, for example, a protein.

FIG. 2 is a schematic representation of a staggered strategy to improve the coverage of protein sequences using bottom-up (small peptides, light grey fragments; left-most two protein representations), middle-down (large peptides, dark grey fragments, right-most two representations), and top-clown approaches (intact protein, black fragments; middle representation).

FIG. 3 is an example of common peptide fragmentation ions produced by mass spectrometry, where (a) and (x) ions are produced by collision induced dissociation (CID) (dotted lines; labeled a₁/x₃, a₂)/x₂, and a₃/x₁); (b) and (y) ions are produced predominantly by collision induced dissociation (CID) (short-dashed lines; labeled b₁/y₃, b₂/y₂, and b₃/y₁), (c) and (z) ions are produced predominantly by electron transfer dissociation (ETD) (long dashed lines; labeled c₁/z₃, c₂/z₂, and c₃/z₁).

FIG. 4 is a schematic representation showing an approach for analyzing a protein containing disulfide bonds. Polypeptides P1 and P2 each contain an inter-chain disulfide bond denoted (1), and are connected through two inter-chain disulfide bonds denoted (2) and (3). The protein without reduction is digested with enzymes to produce disulfide-free peptides and three disulfide-linking peptides DSLP(1), DSLP(2), and DSLP(3). DSLPs then are analyzed by EID-MS², CRCID, and CID-MS³, and disulfide free peptides are fragmented by CID-MS²and CID-MS³.

FIG. 5 is a schematic representation of an exemplary process for characterizing a disulfide knot containing protein. A protein containing a disulfide knot is treated with a single enzyme (Enzyme A) and multiple enzymes, for example, Enzymes (B, C), (B, C, D), and/or (B, C, D, E) with no reduction and/or partial reduction. Disulfide free peptides and disulfide knot containing peptides (DSKCPs) then are analyzed using CID (CID-MS²), ETD (ETD-MS²), and CRCID (CID-MS³).

FIG. 6 is a schematic representation of an exemplary process for characterizing an N-linked glycosylated protein. Process 1 is a deglycosylation process using PNGase F to free N-linked glycans from glycoprotein (Step 1). The N-linked glycans (A) are separated from an intact protein (B) using molecular weight cut-off filters (Step 2). The isolated glycans (A) are subjected to glycan analysis (Step 3). Process 2 using multiple enzymes including PNGase F (Step 1) generates nonglycosylated and deglycosylated peptides (C) to be used for peptide sequencing by CID-MS², CID-MS³, and HCD-MS² (Step 2). Process 3, Step 1 uses multiple enzymes without PNGase F to produce N-linked glycosylated and nonglycosylated peptides (D). N-linked glycosylated peptide sequencing and glycan composition analysis is performed using HCD-MS²/MSⁿ, and CID-MS²/MSⁿ (Step 2). Non-glycosylated peptide sequencing is carried out using CID-MS² CID-MS³, and HCD-MS² in Step 3. N-linked glycosylation sites are confirmed as results of peptide mapping in Process 2/Step 2 as well as Process 3/Step 2 and Step 3.

FIG. 7 (A) is a schematic representation of an exemplary process for characterizing an O-linked glycosylated protein. Process 1 employs multiple enzymes including PNGase F to produce non-glycosylated and O-linked glycosylated peptides in Step 1. O-linked glycosylated and non-glycosylated peptides then are monitored in Step 2 and Step 3, respectively, based on their predicted masses. O-glycosylated peptides are sequenced by ETD-MS², CRCID (CID-MS³), CID-MS², and/or HLCD-MS² (Process 1/Step 5). The site-specific O-glycans is determined using CID-MS² and/or HCD-MS² (Process 1/Step 4). The non-glycosylated peptide sequencing is carried out using CID-MS² nd/or HCD-MS² in Step 6. As results of Steps 5 and 6 in Process 1, O-linked glycosylation sites are determined. Process 2 is deployed for glycan analysis. The Step 1 in Process 2 uses chemicals to release all glycans from a glycoprotein. Released glycans are isolated from an intact protein using molecular weight cut-off filters (Process 2, Step 2). Collected glycans are subjected to glycan analysis (Process 2/Step 3).

FIG. 7 (B) is a schematic representation of an alternative process for characterizing an O-linked glycosylated protein. Process 1 employs multiple enzymes including PNGase to produce non-glycosylated and O-linked glycosylated peptides in Step 1. O-linked glycosylated and non-glycosylated peptides then are monitored in Step 2 and Step 3, respectively, based on their predicted masses. O-glycosylated peptides sequences and site-specific O-glycans are determined by HCD-MS²/MSⁿ, and/or CID-MS²/MSⁿ (Process 1/Step 4) (this combines Process 1/Step 4 and Process 1/Step 5 of FIG. 7(A)). The non-glycosylated peptide sequencing is carried out using CID-MS², CID-MS³, and/or HCD-MS² in Step 6. As results of Steps 4 and 6 in Process 1, O-linked glycosylation sites are determined. Process 2 is deployed for glycan analysis. The Step 1 in Process 2 uses chemicals to release all glycans from a glycoprotein. Released glycans are isolated from an intact protein using molecular weight cut-off filters (Process 2, Step 2). Deglycosylated peptides sequences are determined by HCD-MS², CID-MS², and/or CID-MS³ (Process 2/Step 3). Collected glycans are subjected to glycan analysis (Process 2/Step 4).

FIG. 8 is a schematic representation of an exemplary glycan analysis. Quantitative measurement is achieved by LC-FD-MS by adding fluorescent dye onto the reducing end of glycans (Derivatized Glycans¹). Various derivation methods are conducted as appropriate (Derivatized Glycans²). CID (CID-MS², CID-MS³, and CID-MSⁿ) and FWD (HCD-MS², HCD-MSⁿ) in both positive and negative ion mode are applied to glycan molecules (underivatized and/or derivatized) to characterize structures.

FIG. 9 is an exemplary profile of common fragment ions produced by mass spectrometry. CID and HCD can break glycosidic bonds to generate B, C, Y, and Z ions (dashed lines (- - - )) as well as cross-rings to produce A and X ions (dotted lines (. . . )). D ions can be produced by breaking two glycosidic bonds (B and Y) at the branch (dot-dash lines (.-.-)).

FIG. 10 is a schematic representation of an exemplary process for generating an in vivo comparability profile. In Steps 1 and 1′, a reference biologic (depicted as the lighter molecule) or candidate biologic (depicted as the darker molecule) is administered to a subject, in Steps 2 and 2′, the biologic and optionally one or more of its metabolites are extracted from the subject. In Steps 3 and 3′, the biologic and optionally one or more of its metabolite are fragmented, and in Step 4. the resulting fragments are subject to mass spectroscopic analyses. Categories of the analyses' resulting output are provided. It is understood that Steps 1, 2, 3 and 4 can be performed. before, contemporaneous with, or after Steps 1′, 2′, 3′, and 4′.

FIG. 11 is a schematic representation of the exemplary process depicted in FIG. 10, further depicting exemplary mass spectroscopic analysis output data.

FIG. 12 is a schematic representation of an exemplary feature or range measurement for in vivo comparability profile assessment, which depicts the change in the ratio of relative abundance of a feature or group of features over time between a candidate and reference biologic.

FIG. 13 (A) and FIG. 13(B) depict an exemplary cluster analysis/cluster graph (FIG. 13(A)) and an exemplary peak profile analysis (FIG. 13(B)), both of which depict the results of statistical analysis of mass spectroscopic data from the reference and candidate biologics, and which may be used to demonstrate in vivo comparability between the reference and candidate biologics.

FIG. 14 is a schematic representation of two exemplary processes for generating in vivo comparability profiles comparing either a native protein (I) or reference biologic (II) to the expression product of a gene therapy construct that is expressed in cell culture (III), or in a subject (IV). In Step 1 the reference biologic (depicted as the lighter molecule) is administered to the subject, and in step 2, the reference biologic and optionally its metabolites are extracted. As shown in (I), if the reference is the endogenous protein, then the administration step (Step 1) is omitted, and the protein is extracted in Step 2. In Step 1′, the gene therapy construct is administered to cells in culture (III) or to a subject (IV), and in Step 2′, the resulting transcription product is extracted. In Steps 3 and 3′ the extracted biologics are fragmented, and in Step 4, the fragments are subject to mass spectroscopic analyses. Exemplary results of the mass spectroscopic data analyses are provided. It is understood that the native protein (I) or the reference biologic (II) may serve as the reference for either the expression product of a gene therapy construct expressed in cell culture (III), or in a subject (IV). It is further understood that Steps 1, 2, 3 and 4 can be performed before, contemporaneous with, or after Steps 1′, 2′, 3′, and 4′.

Other aspects, embodiments, and features will be become apparent from the following detailed description when considered in conjunction with the accompanying figures.

### DETAILED DESCRIPTION

There is a need for a systematic process for characterizing biologic drugs after they have been administered to a subject. In some cases, the biologic drug is modified, for example, via proteolytic cleavage following exposure to proteolytic enzymes, subjected to oxidation, reduction, or deamination reactions, or subjected to post translational modifications such as glycosylation or deglycosylation as the case may be, etc. There is a need for developers, manufacturers, and, in some cases, regulators, of biologic dugs to assess how a candidate biologic drug structurally changes over time in vivo as compared to a reference biologic drug given that small differences in structure combine profound effects in the stability and/or function of a molecule. This type of information can be used to generate an in vivo comparability profile, which can then be used to assess the comparability of the candidate biologic drug and the reference biologic drug. A detailed profile of the structure of the biologic, complete with identification of the features modified during its residence in a subject provides a tool that enables more effective and accurate development of innovator biologics, biosimilars, and gene therapy constructs.

The invention provides methods for determining a set of molecular structural features or attributes of a biologic drug that, as the case may be, may or may not be modified in vivo following administration to a subject. The invention provides data that can be computationally or statistically evaluated either by individual structural feature or by groups of such features in comparability testing of molecules. Practice of the process can result in a compilation of information that may serve as a profile of molecular features (for example, structural features) modified during in vivo residence, which serve as critical indicators of the drug's behavior in vivo. This structural information can be compared to in vitro structural information, and may be combined with functional data, if or when available, for example, via one or more functional assays, which may demonstrate whether a structural change has a major effect, modest effect or no material effect on the activity of the candidate molecule and/or the reference molecule or a respective metabolite thereof. The resulting in vivo comparability profile can serve as a development tool, e.g., as a guide or target during the development biosimilars or gene therapeutics, or as a definitive identifier of the specific biologic. It is contemplated that the profile may serve as the definition of the reference biologic and provides a means by which a given product may be reliably reproduced and/or characterized as suitably similar to another biologic product or biosimilar to another biologic product.

For example, an in vivo comparability profile can be used in the development, testing, and manufacture of, for example, innovator biologic drugs based upon, for example, an endogenous molecule. Similarly, an in vivo comparability profile can be used in the development, testing and manufacture of biosimilars and highly similar biosimilars when compared to a previously approved biologic drug. Furthermore, the in vivo comparability profile can be used to test lot-to-lot variability during manufacturing based on how different batches of a biologic drug behave in vivo. Furthermore, the in vivo comparability profile can be used to test how gene expression products produced from an expression construct that is delivered for example, via gene therapy, behave relative to a molecule whose expression or activity is meant to the modulated by gene therapy.

In one aspect, the invention provides a method of measuring in vivo comparability of a candidate biologic drug to a reference biologic drug. The method comprises the steps of: (a) generating through mass spectroscopic analysis data indicative of the structure of the candidate biologic drug or a metabolite thereof following extraction from a sample, for example, a cell, tissue or body fluid sample, removed from a subject, for example, a human or animal, at a first time interval following administration of the candidate biologic drug to the subject; and (b) comparing the data generated during step (a) to mass spectroscopic analysis data indicative of the structure of the reference biologic drug or a metabolite thereof generated through analysis of a sample taken at the same time interval from a subject to whom the reference biologic drug had been administered, thereby to produce an in viva comparability profile of the candidate biologic drug to the reference biologic drug.

It is contemplated the mass spectroscopic analysis data indicative of the structure of the reference biologic drug or a metabolite thereof can be generated before, after, or contemporaneously with, the generation of the mass spectroscopic analysis data indicative of the structure of the candidate biologic drug. It is contemplated, however, that the mass spectroscopic data are generated on samples that have been treated in the same manner, for example, administered by the same route of administration, harvested in the same manner and after similar residence times in the subject, and, when appropriate, purified, in the same way, and analyzed in the same manner.

The data indicative of the structure of the candidate molecule or the metabolite thereof can comprise a normalized measure (or measurement) of a modified form of a structural attribute of interest, wherein the normalized measure corresponds to the amount of a modified form of the structural attribute of interest relative to the total amount of the structural attribute (i.e., the sum of modified attribute and unmodified attribute) in the sample being tested. For example, a normalized measure of a modified structural attribute (for example, the levels of one or more major glycoforms, deamidated or oxidized moieties, disulfide or trisulfide bonds) can be calculated, for example, by dividing the amount of, for example, a peptide containing the modified structural attribute of interest by the sum of the amount of all of the corresponding peptides (the sum of the amount of peptides containing the modified structural attribute and peptides containing, the unmodified structural attribute) in the sample. The results can be presented in any appropriate form, for example, as a ratio, percentage, etc.

The comparability profile comprises one or more sets of data indicative of: (a) a structural feature that is the same or different when comparing the candidate biologic drug or a metabolite thereof and the reference biologic drug or a corresponding metabolite thereof; and optionally, when available, (b) data indicative of whether differences in a structural feature between the candidate biologic drug and the reference biologic drug substantially affect a biological activity of the candidate biologic drug. The resulting data can be used to create a record of structural features of the candidate molecule and/or reference molecule, for example, a profile, which identifies and characterizes parts of the molecular structure that are modified during in vivo residence. The profile can reveal which particular attributes or modifications affect the molecule's stability and/or activity, and which attributes or modifications do not materially affect stability and/or activity. The profile can provide evidence of how the biologic drug (the candidate drug, the reference drug or both) is processed within the subject, and thus can serve as a comparison of how the candidate molecule (for example, biosimilar) and the reference molecule (for example, the approved biologic) behave in vivo following administration to a subject.

The mass spectrometry analysis data can be generated by fragmenting or chemically modifying, as appropriate, the reference biologic drug, the candidate biologic drug, or metabolites thereof before or while subjecting the sample to mass spectrometric analysis. Depending upon the molecules in question, mass spectrometry may be conducted on one or more extracted species selected from the group consisting of derivatized, truncated, oxidized, methylated, deaminated, aggregated, differentially glycosylated, improperly disulfide bonded, structurally intact protein species or fragments thereof, or a combination of such extracted species.

By exploiting recently developed, known analysis techniques, the invention contemplates that the candidate molecule and/or the reference molecule can be characterized by one or more of, for example, its primary, secondary, tertiary and/or quaternary structures. The mass spectroscopic analyses can determine, without limitation, the precise post-translational peptide sequence, the pattern of inter and intra-chain disulfide bonding including the presence of any disulfide knots, whether and where the molecule is glycosylated the pattern of the glycosylation, whether the molecule is truncated after expression, whether its activity is dependent on covalent or non-covalent dimerization, and generally, the nature of its post-translational modification(s).

The generation of mass spectrometry data can be obtained using one or more techniques selected from the group consisting of electron transfer dissociation mass spectrometry, collision induced dissociation mass spectrometry, higher-energy collisional dissociation mass spectrometry, electron capture dissociation mass spectrometry, infrared multi-photon dissociation mass spectrometry, ultraviolet photodissociation mass spectrometry, hydrogen/deuterium exchange mass spectrometry, MS², MS³, and LC-MS. Tandem mass spectrometry data can be acquired using techniques selected from the group consisting of data-dependent acquisition, data-independent acquisition, selected reaction monitoring, and parallel reaction monitoring. In addition, the mass spectrometry analysis data can be generated by one or more analytical techniques selected from the group consisting of fluorescent spectra, light scattering spectroscopy, electrophoresis, selective proteolysis, UV spectra analysis, IR spectra analysis, Hydrogen-Deuterium exchange analysis, and MRI spectra analysis.

This step results in the identification of substructures of the molecule that is modified during in vivo residence, i.e., identify and characterize parts of the molecular stricture modified, degraded, or otherwise changed following administration to a subject. Thereafter, it may be determined which of the potentially labile features are material to efficacy, safety, or potency (and inferentially, those which are not material to efficacy, safety or potency).

The comparability profile generated in step (b) may be of sufficient detail to permit qualification of the candidate biologic drug as (i) biosimilar to the reference biologic drug, (ii) as substantially the same as the reference biologic drug, or (iii) interchangeable with the reference drug. Where the candidate biologic drug and the reference biologic correspond to different manufacturing lots of the same biologic drug, the comparability profile generated in step (b) may of sufficient detail to serve as criteria to qualify the release of the manufacturing lot containing the candidate biologic drug.

In certain embodiments, the in vivo comparability profile can be used to facilitate the production of innovator biologics. For example, a candidate biologic drug can be developed using various different raw materials, expression conditions, purification techniques and reagents until a biologic product is produced which has an in vivo comparability profile that shows that the biologic drug being developed has similar structural features (and optional functional attributes) as the endogenous reference protein. Furthermore, the in vivo comparability profile can be used to facilitate the development of a biosimilar. For example, a biosimilar can be developed using various expression constructs, expression conditions, purification techniques and reagents until a biosimilar is produced which has an in vivo comparability profile that shows that the biosimilar has similar structural features (and optional functional attributes) as the approved reference biologic.

In certain embodiments, the profile can have sufficient detail to serve as criteria to qualify for regulatory purposes a biologic drug as biosimilar to, or interchangeable with, another previously marketed biologic drug approved by a regulatory agency. The method comprises analyzing the biosimilar to assure that its attributes (structural and optional function attributes) indicate that it is metabolized or otherwise modified or broken down in vivo in the same way as the previously approved biologic drug, such that it satisfies the in vivo comparability profile produced in accordance with the method described above. The information can be used to support an approval that the candidate molecule is biosimilar or even highly similar to the reference molecule.

Furthermore, the in vivo comparability profile can be used to facilitate the development of gene therapy-based approaches to replace or augment an endogenous protein. For example, an expression construct can be developed, modified, as appropriate, and tested to produce an expression product (for example, protein) that has an in vivo comparability profile that has similar structural features and optional functional attributes) as the endogenous molecule (for example, endogenous protein) that is intended to be augmented or modulated by the gene therapy.

The following sections discuss the preparation and characterization of the candidate biologic as well as the reference biologic, and the identification of structural features or attributes that are modified during in vivo residence, which may be important to the safety, and/or potency biological molecule.

### I. Definitions

As used herein, the terms “biologic” and “biologic drug” are understood to mean a drug or drug, candidate that is produced by recombinant DNA technologies, peptide synthesis, or purified from natural sources and that has a desired biological activity. The biologic or biologic drug can be, for example, a protein, peptide, glycoprotein, polysaccharide, a mixture of proteins or peptides, a mixture of glycoproteins, a mixture of polysaccharides, a mixture of one or more of a protein, peptide, glycoprotein or polysaccharide, or a derivatized form of any of the foregoing entities. The molecular weight of biologics can vary widely, from about 1000 Da for small peptides such as peptide hormones to one thousand kDa or more for complex poly saccharides, mucins, and other heavily glycosylated proteins. The biologic subject of the process of tins invention can have a molecular weight of 1 kDa to 1000 kDa, more typically 20 kDa, to 200 kDa, and often 30 kDa to 150 kDa. By way of example, desmopressin, oxytocin, angiotensin and bradykinin each have a molecular weight of about 1 kDa, calcitonion is 3.5 kDa, insulin is 5.8 kDa, kineret is 17.3 kDa, erythropoietin is about 30 kDa, ontak is 58 kDa, orencia is 92 kDa, and antibodies are approximately 150 kDa (Rituxan 145 kDa, Erbitux 152 kDa). Hyaluronic acids and salts have an average molecular weight often greater than 1000 kDa. Examples of biological drugs include, for example, native or engineered antibodies or antigen binding fragments thereof, and antibody-drug conjugates, which comprise an antibody or antigen binding fragments thereof conjugated directly or indirectly (e.g., via a linker) to a drug of interest, such as a cytotoxic drug or toxin. As used herein, the terms “reference biologic” and “reference biologic drag” refer to a biologic that is representative of the biologic drug under development or that that has been approved for marketing, and provides a reference standard for the biologic drug with, for example, the appropriate, pre-determined composition, purity and/or biological activity.

As used herein, the term “subject” is understood to mean a human, or a non-human animal, for example an experimental animal.

As used herein, the terms “assessing” is understood to mean measuring, determining, or quantifying.

As used herein, the term “in vivo comparability” is understood to mean the degree of structural similarity between two biologic molecules and their metabolites after a period of in vivo residence in a subject. As used herein, the term “in vivo comparability profile” is understood to mean a compilation of comparative data (including statistically processed data) indicating a structural feature or set of structural features of a biologic molecule and/or one or more of its metabolites after a period of in vivo residence in a subject when compared to the same structural feature or set of structural features in a reference molecule and/or one or more of its metabolites.

As used herein, the term “biosimilar” means of sufficient similarity to qualify as a biosimilar according to a regulatory agency, for example, the U.S. Food and Drug Administration (“USFDA”) or other regulatory agencies.

As used herein, the term “interchangeable” means of sufficient similarity to qualify as an interchangeable biosimilar according to a regulatory agency, for example, the USFDA or other regulatory agencies. It is understood that a biologic drug qualifying as interchangeable with a reference biologic drug is also biosimilar to the reference biologic drug.

As used herein, the term “substantially the same” is understood to mean of sufficient similarity to qualify as substantially the same in terms of safety, purity, potency or a combination thereof or release criteria for quality assurance purposes.

As used herein, the term “qualify the release of” means of sufficient similarity to qualify the release of a batch or lot of biologic drug.

### II. Administration of Biologics

### A. Administration of Biologic Produced Outside of the Intended Recipient

It is contemplated that the biologic, for example, a protein, may be administered to a subject using or more approaches, for example, via oral administration, via intravenous, intramuscular, or subcutaneous injection, or by other methods that are readily known to one of skill in the art.

### B. Administration of a Biologic Produced Inside of the Intended Recipient

It is contemplated that a biologic of interest may be the expression product of a gene therapy protocol. For example, a nucleic acid, such as a gene therapy expression construct, is administered to the subject, and the nucleic acid is subsequently transcribed and translated if the gene therapeutic is DNA-based, or translated if the gene therapeutic is RNA-based, to produce the protein of interest, or can be the expression product of a gene modified by the CRISPR/CAS9-based gene editing system. It is understood that the gene therapy expression constructs may be administered by any of a number of ways known to those skilled in the art, any of which are appropriate for use with the instant invention, provided that the gene therapeutic results in the production of an expression product.

### III. Structural Characterization of Candidate and Reference Biologic

### A. Sample Extraction, Purification, and Preparation

The structure of biological molecules can be very complex. With proteins or glycoproteins, for example, any modification to the backbone amino acids (for example, by deamidation, oxidation, methylation, acetylation, and isomerization etc.), changes in disulfide bonds (for example, shifting to cause mispaired cysteine bridges or unpaired cysteine residues), changes in glycosylation (for example, glycosylation site and glycan pattern) or the post translational modification can cause changes in protein folding or stability leading to loss of biological function. As a result, it may be necessary to extract, purity, process, and analyze the biologic drug or reference drug using techniques appropriate each particular molecule and its metabolites.

1. Extraction

The method of extraction of a biologic of interest will vary depending upon whether the sample is a cell, tissue or body fluid sample. If the biologic of interest is present in the blood, extraction may be effectuated by removing a sample of blood from the subject. For other biologics, it may be necessary to perform a biopsy, for example a muscle biopsy. It is contemplated that, for a given biologic of interest, samples may be taken from multiple physiological compartments in order to obtain data showing the biologic's metabolism throughout the body. Relevant physiological compartments may be selected based on how the biologic is expressed, transported within the recipient, and metabolized.

2. Purification

It is contemplated that, for a biologic of interest, the candidate and reference biologic molecule preferably has a purity of at least 50%, i.e., the molecule comprises at least 50% by weight of all of the components in a given sample. If the purity is less than 50%, the molecule preferably is purified to remove impurities (for example, process-related contaminants, unrelated biological macromolecules, misfolded proteins, etc.) using conventional purification techniques known in the art. For example, the molecule can be purified from other macromolecules present in a cell sample (for example, unrelated nucleic acids, lipids, glycolipids, polysaccharides, lipopolysaccharides, proteins, or even misfolded and/or misprocessed forms of the biological molecule of interest) (Conlon et al., METHODS MOL BIOL. (2012) 917: 369-390). Conventional purification techniques can be used, which include immunocapture (also known as immunoaffinity) and non-antibody based purification approaches. Immunoaffinity uses an antibody or a ligand that selectively binds the biological molecule of interest (Urh et al., METHODS IN ENZYMOLOGY (2009) Volume 463, Chapter 26, pp. 416-438; Moser et al., BIOANAYLSIS (2010) 2(4): 769-490). This technique can be very effective in selecting the molecule of interest from a complex mixture, for example, an extract.

Non-antibody-based protein purification protocols can include, but are not limited to, protein precipitation (Brugess, METHODS IN ENZYMOLOGY (2009) Vol. 463, Chapter 20, pp. 331-342), gel filtration (Stellwagen, METHODS IN ENZYMOLOGY (1990) Vol. 463, Chapter 23, pp. 373-385), ion exchange chromatography (Iwigbauer et al., METHODS IN ENZYMOLOGY (2009) Vol. 463, Chapter 22, pp. 349-371), and gel electrophoresis (Garfin, METHODS IN ENZYMOLOGY (1990) Vol. 463, Chapter 29, pp. 497-513; Friedman et al., METHODS IN ENZYMOLOGY (2009) Vol. 463, Chapter 30. pp. 515-540). Protein precipitation methods utilize solvents (for example, ammonium sulfate, polyethyleneimine, acetone, acetonitrile, or ethanol, etc.) to remove unwanted small molecules by precipitating the molecules of interest and/or by precipitating unwanted molecules. Size-based fractionation can be achieved by gel filtration (for example, by size-exclusion chromatography) where molecules are separated based on differences in molecular size during passage through a porous medium packed in a gel filtration column. Ion exchange chromatography utilizes charge differences between a stationary phase and a mobile phase to purify the molecule of interest. For example, positively charged proteins can be isolated using a cation exchange chromatography where the stationary phase is negatively charged. Negatively charged proteins can be separated using art anion exchange chromatography in which stationary phase is positively charged. Gel electrophoresis, for example, one-dimensional gel electrophoresis (Garfin (1990) supra), or two-dimensional gel electrophoresis (Friedman (2009) supra) fractionates molecules based on size, shape, and charge. The molecule of interest, such as, a monoclonal antibody, can be isolated by sodium dodecyl sulfate (SDS) polyacrylamide gel under non-reducing and/or reducing conditions. It is understood that a number of purification techniques may be used in combination to achieve the requisite purity.

After fractionation, the molecules can be visualized by standard staining approaches (for example, using SimplyBlue™ SafeStain or SilverQuest™ Silver Staining Kit, both available from. Life Technologies). The unbound stain can be removed by rinsing the gel with deionized water and/or ammonium bicarbonate buffer until the gel has no stain (for example, blue stain) background color. The gel bands of interest, once identified, are cut from the gel and dried with acetonitrile using a CentriVap vacuum concentrator. The molecules in the gel bands can be harvested from the gel band or treated or analyzed further within the gel, for example, exposed to in-gel digestion.

3. Denaturation

During the characterization process, the molecules of interest may be denatured under a variety of conditions where the molecules unfold and lose their secondary, tertiary, and/or quaternary structures. The primary structure of protein is based upon the amino acid. sequence of the protein. Functional proteins typically are fold into a high order structure involving secondary structures (for example, via hydrogen bonding between peptides to form α-helices and β-sheets), tertiary structures (for example, through hydrophobic interactions, ionic interactions, and disulfide bridges to produce a three-dimensional protein molecule), and quaternary structures (for example, by non-covalent interactions and disulfide bonds to connect subunit proteins) (Tones, ADVANCED DRUG DELIVERY REVIEWS (1993) 10:29-90).

In order to characterize higher order structures, the molecule of interest can be subjected to denaturation, which disrupts secondary, tertiary, and quaternary structures. Denaturation can involve exposure to elevated temperatures (Freire, METHODS IN ENZYLMOLOGY (1995) 259: 144-168; Friedman (2009) supra), exposure to chemical denaturants (Neurath et al., CHEM. REV. (1944) 34(2): 157-265; Makhatadze et al., J. MOL. BIOL. (1992) 226: 491-505), and exposure to mechanical stress, for example, freeze-thaw processes (Pikal-Cleland et at, ARCHIVES OF BIOCHEMISTRY AND BIOPHYSICS (2000) 384(2): 398-406).

Thermal denaturation (for example, heat at 100° C. for 10 minutes), can disrupt hydrogen bonds occurring between amide groups in the secondary protein structure and hydrophobic interactions in the tertiary protein structure. Chemical denaturation may include the use of organic solvents (for example, cleavage of hydrogen bonds in secondary and tertiary structures by acetonitrile, methanol, or ethanol), acids (for example, dissociation of ionic interactions by formic acid, trichloroacetic acid, hydrochloride), and chaotropic agents (for example, disruption of hydrophobic interactions in tertiary and quaternary structures by urea, guanidine hydrochloride, sodium dodecyl sulfate). Mechanical stress for example, freezing-thawing process) can also induce protein denaturation in solution or lyse a cell sample by disrupting the cell through ice crystal formation.

4. Reduction and Alkylation

The molecule of interest may contain a disulfide bond (S—S) (also known as a disulfide bridge), that is formed by coupling two thio groups (—SH) from cysteine residues (Mullan et al., BMC PROCEEDINGS (2011) 5 (Suppl 8): 110). The disulfide bridges can include intra- and/or inter-chain disulfide bonds. For example, monoclonal antibodies comprise two light (L) chains and two heavy (H) chains that are covalently linked by inter-chain disulfide bonds. in addition, intra-chain disulfide bonds can be found in variable (V) and constant (C) domains on each light and heavy chain. Mispaired disulfide bonds including unpaired cysteines (for example, occurring to pH change during manufacturing purification process or storage) can result in loss of biological activity such as drug efficacy, for example, as typically occurs with monoclonal antibodies. Therefore, it is important to determine the location of intra-chain and inter-chain disulfide bonds and their status (for example, unpaired, mispaired, or scrambling).

Disulfide bridges can be cleaved using reducing agents such as 2-mercaptoethanol (Jocelyn, METHODS IN ENZYMOLOGY (1987) 143:246-255), dithiothreitol (DTT) (Jocelyn (1987) supra), and tris(2-carboxyethy)phosphine (TCEP) (Getz et al., ANALYTICAL BIOCHEMISTRY (1999) 273: 73-80). For example, during a reduction reaction, the molecule of interest can be subjected to a solution containing 50 nM DTT at 37° C. for 30 minutes to break cysteine bridges by reducing the disulfide bonds. To prevent reformation of disulfide bonds after protein reduction, opened cysteine residues (or called free thiol groups, —SH) are capped using an alkylating agent such as iodoacetic acid, iodoacetamide (IAA), or N-ethylmaleimide (NEM) (Anfinsen et al., J. BIOL. CHEM (1961) 236: 1361-1363). For example, 100 mM IAA is commonly added to protein solution after completion of DTT reduction and then kept in dark at room temperature for 45 minutes.

5. Fragmentation

Depending upon the characterization methods to be used, the molecule of interest can be fragmented using a variety of techniques, which can be executed in solution (solution digestion), in a gel (in-gel digestion), or in a gas phase (gas-phase fragmentation). In general, enzymes and chemicals can be utilized to cleave molecules, for example proteins, in solution and in gel. Electric forces can be induced in the gaseous phase of an electronic instrument (for example, a mass spectrometer) to fragment molecules of interest.

### a. Enzymatic Digestion in Solution

Exemplary proteolytic enzymes include trypsin, Lys-C, Asp-N, and Arg-C, in which cleavage sites are highly specific. Exemplary deglycosylating enzymes release glycans from glycoproteins, which can include N-glycosidases such as peptide N-glycosidase F (PNGase F), O-glycosidase sialidase, glucosaminidase, and β-galactosidase etc. (Jensen et at., NATURE PROTOCOLS (2012) 7(7): 1299-1310). These enzymes are commonly used to generate various sizes of peptides and/or oligosaccharides. For examples, trypsin cleaves the carboxyl side of lysine (Lys) and arginine (Arg) residues if the next residue is not proline (Pro). Lys-C hydrolyzes the peptide bond at the carboxyl side of Lys. Glu-C cuts the carboxyl side of glutamate (Glu). Arg-C cleaves the C-terminal side of Arg residues including the site next to Pro. Asp-N breaks peptide bonds on the N-terminal side of aspartic acid (Asp) residues. PNGase F hydrolyzes the amide bond of asparagine (Asn) to Asp residues and releases oligosaccharides from N-linked glycoproteins. Less specific enzymes such as pepsin, papain, chymotrypsin, aminopeptidases, carboxypeptidases can also be used to produce fragments depending of the structural complexity of interest. (Switzar et al., J. PROTEOME RES. (2013) 12: 1067-1077). For example, pepsin cleaves monoclonal antibodies (mAb) into F(ab)₂ fragment and papain typically breaks mAb into two Fab fragments and an intact Fc fragment.

To obtain a full coverage of a molecule structure of interest, it can be necessary to use several enzymes, which can be introduced as single enzymes in a series of separate digestions or can be used as a mixture of enzymes.

For multiple single enzymes in a serial process, for example, trypsin, is added to a protein solution (1:50 w:w) in 100 mM ammonium bicarbonate or 100 mM tris-HCl buffer (pH 6.5 to 8), and incubated at room temperature or at 37° C. After 4-hour incubation, trypsin can be added to a protein solution (1:50 w:w) again and incubated for another 16 hours. Formic acid, 5%, can be used to stop the enzymatic reaction. Then, the trypsin digest is subjected to exchange buffer with 10 mM HCl (pH 2) before pepsin digestion (pepsin: protein 1:10 w:w) is conducted at 37° C. for 30 minutes. The pepsin reaction can be terminated by adjusting pH to 5 with ammonium bicarbonate buffer. The resulting digest then is subjected to a buffer exchange with 100 mM ammonium bicarbonate (pH 8) if the digestion solution is subjected to another digestion. For example, PNGase F (1 unit/10 μg) is added to pepsin digestion solution after buffer exchange. Deglycosylation reaction is carried out at 37° C., up to 24 hours. Formic acid, 5%, can then be added to stop the enzymatic reaction.

Alternatively the molecule of interest can be exposed to multiple enzymes in a single reaction mixture if digestion conditions such as optimal pH ranges for each individual enzyme are similar, for example, a cocktail mixture of (trypsin and Lys-C), (trypsin, Lys-C, Asp-N), or (trypsin, Lys-C, Asp-N, PNGase). If pepsin is added for multiple enzyme digestion, a serial process is required. This is because pepsin is only active under an acidic condition (pH 2). For example, if a solution containing a monoclonal antibody is subjected to pepsin digestion in 10 mM HCl (pH 2, 37° C., 30 minutes) buffer should be exchanged, for example, with 100 mM ammonium bicarbonate at room temperature using 10,000 molecular weight cut-off membrane filters, before continuing the digestion with trypsin (1:50 w:w) and/or Lys-C (1:50 w:w).

### b. Chemical Digestion in Solution

Chemicals useful in fragmenting proteins include cyanogen bromide (CNBr) (Zhang et at., ANAL. CHEM (1996) 68(19): 3422-3430), 2-nitro-5-thiocyanobenzoate (NTCB) (Tang et al., ANAL. CHEM. (2004) 334: 48-61). hydroxylamine (Burnstein et al. METHODS ENZYMOL. (1977) 47: 132-45), and formic acid (FA) (Landon, METHODS IN ENZYMOLOGY (1977) 47: 145-149) etc. CNBr breaks peptide bonds at the C-terminal side of methionine (Met) residues. NTCB cleaves proteins at cysteine (Cys) residues through reactions of cyanylation and β-elimination. Hydroxylamine hydrolyzes asparaginyl glycyl (Asn-Gly) peptide bonds. Formic acid cuts proteins at aspartic acid-proline (Asp-Pro) peptide bonds.

Similar to enzymatic digestion, multiple chemicals can be included in the same reaction mixture provided that the reaction conditions are compatible with each chemical. For example, CNBr reaction is often coupled with 70% formic acid or 70% trifluoroacetic acid (TFA) in dark at room temperature for 16 hours (Zhang (1996) supra).

To increase coverage for structural characterization, a combination of enzymes and chemicals can be subjected to protein of interest (Kwon, A JOURNAL OF CHROMATOGRAPHY (2010) 1217: 285-293). For examples, proteins can be submitted to acid hydrolysis using 25% formic acid and incubated at 95° C. for 4 hours. After buffer exchange with 100 mM ammonium bicarbonate (pH 8), the protein solution can be further treated by a single enzyme or multiple enzymes.

Fragments obtained from solution digestion can be directly submitted for structural analysis by mass spectrometry. However, extraction (for example, solid phase extraction or liquid/liquid extraction) may be required to improve the recovery of peptides generated from solution digestion.

### c. In-Gel Digestion with Enzymes and/or Chemicals

The molecules of interest, when captured in a gel band, can be subjected to digestion in situ. For example, the gel bands of interest are cut into approximately 1×1 mm slices, and then are subjected to digestion (in-gel digestion). Similar to solution digestion, enzymes and/or chemicals can be used to cleave proteins of interest in each gel slice. In-gel digestion can be conducted with or without in-gel reduction and alkylation (Shevchenko, NATURE PROTOCOLS (2006) 1(6): 2856-2860). For example, if a gel band is excised under a non-reducing condition, dried gel pieces are covered with 10 mM DTT in ammonium bicarbonate buffer and incubated at 56° C. for 30 minutes. Then 55 mM IAA is added to cover the gel pieces in dark at room temperature for 45 minutes after removing the remaining DTT solution. The reduced and alkylated gel samples then are dried with acetonitrile using a CentriVap vacuum concentrator, followed by washing the samples with ammonium bicarbonate buffer and deionized water. Ammonium bicarbonate buffer containing enzymes and or chemicals is added to cover the dried gel bands. The remaining buffer containing enzymes and/or chemical is removed after incubating at 4° C., up to 1 hour. Blank buffer without enzymes or chemicals then is added to cover the gel samples and incubate at 37° C. for overnight.

Fragments obtained from in-gel digestion are typically extracted with 5% formic acid and acetonitrile (1:2 v:v) and then dried down using a CentriVap vacuum concentrator to yield approximately 10 μL of wet extract for each gel digest prior to mass spectrometric analysis.

### B. Characterization of the Biologic

Mass spectrometry is the most suitable and useful tool to be used for characterization of complex biologics (Mann, ANNU. REV. BIOCHEM. (2001) 70: 437-473; Jardine, METHODS IN ENZYMOLOGY (1990) 193: 441-455). This is because mass spectrometers directly read the mass fingerprints (mass/charge ratios, m/z) of intact or fragmented proteins or molecules. Four types of mass spectrometers, quadrupole, ion trap, time-of-flight (TOF), and fourier transform ion cyclotron resonance (FTICR), have been widely used to obtain structural information for biomolecules. Modem hybrid mass analyzers such as a hybrid linear ion trap-obitrap (e.g., Thermo LTQ Obitrap Elite) and a quadrupole-time-of-flight (e.g., Agilent Q-TOF) have been developed for structural characterization of biologics to support biopharmaceutical discovery and development pipelines. Fragmentation techniques (gas-phase fragmentation) used on mass analyzers include collision induced dissociation (CID), higher-energy collisional dissociation (HCD), electron capture dissociation (ECD), electron transfer dissociation (ETD), infrared multi-photon dissociation (IRMPD), ultraviolet photodissociation mass spectrometry, and CID of the isolated charge-reduced ions followed by ETD (CRCID), depending on the type of mass spectrometers used (Scigelova, PRACTICAL PROTEOMICS (2006) 1-2: 16-21; Elviri, TANDEM MASS SPECTROMETRY—APPLICATIONS AND PRINCIPLES (2012) 162-178). For example. CID, HCD, and ETD are built on a hybrid linear ion trap-obitrap mass spectrometer; CID and ECD are constructed on FTICR MS; low-energy CID is configured on Q-TOF MS; high-energy CID is included on TOF/TOF MS.

Typically, CID can be used for small (for example, peptides 15-20 amino acid residues in length), low charge (for example, +1, +2, +3 charged state) and unmodified peptides. A low-energy CID fragmentation occurs at amide bonds of the peptide bone to generate typically characteristic b and y sequencing ions, which are particularly suitable for peptide sequencing. CID fragmentation depends on the protein or peptide sequence, the peptide length, or the presence of post-translational modifications (PTMs). For example, a peptide having several basic amino acid residues can prevent random protonation on the peptide backbone inducing site specific dissociation and few sequence ions. Certain post-translational modifications can prevent the random protonation on the peptide backbone and subsequently inhibit CID fragmentation.

ECD is based on the gas phase fragmentation of multiple charged protein and peptide ions upon capture of a low energy electron within a mass analyzer such as FTICR MS. The ECD fragmentation can take place through the cleavage of N-Cα bond on the peptide bone to generate c and z ions series of peptide fragments (Elviri (2012) supra) and is able to retain post-translational modifications. However, a large amount of pure sample is required for this approach.

ETD fragmentation involves in a proton-electron transfer process between large peptides or proteins and reagents (for example, transfer an electron from a radical anion to a protonated peptide) (Elviri (2012) supra). Through a proton-electron transfer, peptide backbone N-Cα bonds can be fragmented into c and z sequencing ions without dissociating side chain of amino acid residues. ETD does not require a large amount of sample to be used for MS analysis. Both ECD and ETD are independent of the peptide length and amino acid compositions, which are suitable to fragment an intact protein, large peptides, or labile proteins with post-translational modifications.

To permit the introduction of biologics, such as proteins and peptides, into a mass spectrometer, an electrospray ionization (ESI) or matrix-assisted laser desorption ionization (MALDI) sources are the most common interfaces employed on mass spectrometers. The ESI interface enables on-line introduction of samples (analytes) using HPLC, CE, or infusion pump to deliver analytes from solution phase into gas phase on a mass analyzer. The MALDI interface is especially beneficial for a sample where the amount is limited. For example, a protein or peptide sample (1 μL) typically is spotted on a MALD1 plate having a matrix such as α-cyano-4-hydroxycinnamic acid or sinapinic acid to form a crystal prior to MS analysis. Mass spectrometric methods using ESI ionization with CID, HCD, ETD, and CRCID fragmentation techniques can be used to facilitate structural characterization of biologics.

1. Molecular Weight

Molecular weight is an important aspect of biologics of interest Conventional techniques used to measure molecular weights of proteins include, but not limited to, gel filtration chromatography (“Gel Filtration. Principles and Methods” (2002) supra; Laue et al., METHODS IN ENZYMOLOGY (1990 182:566-587), (or called size exclusion chromatography, SEC), electrophoresis (Jardine (1990) supra; Laue (1990) supra), light scattering (Harding, METHODS IN MOLECULAR BIOLOGY (1994) 22.85-95), analytical ultracentrifugation (Harding, METHODS IN MOLECULAR BIOLOGY (1994) 22:75-84) and mass spectrometry (Jardine (1990) supra; Siliveira (2009) supra; Wysocki (2004) supra; Scigelova (2006) supra; Elviri (2012) supra; Laue (1990) supra), (MS) etc. SEC and gel electrophoresis provide relative molecular weights based upon comparison with molecular weight standards. Absolute molecular weights can be measured by light scattering, analytical ultracentrifugation, and mass spectrometry.

For example, an intact protein (1 mg/mL) in solution after purification can be analyzed directly by a mass spectrometer (for example, Thermo Q Exactive Plus) equipped with an ESI interface to determine molecular weight. The molecular weight of the protein can be obtained after deconvolution of multiple charged states of intact protein using Thermo “Protein Deconvolution” or “PepFinder” software. A combined technique, for example, SDS PAGE-MS (Schuhmacher et al., ELECTROPHORESES (1996) 17:848-854), capillary electrophoresis-MS (Haselberg et al., ELECTROPHORESIS (2011) 32:66-82), or high pressure liquid chromatography-MS (Shi et al., JOURNAL OF CHROMATOGRAPHY (2004) 1053: 27-36), is utilized to determine MWs of biologics if the conditions of biological references are complicated (for example, purity <50%) or not in favor for MS analysis (for example, containing high salts). For example. SDS PAGE can be carried out to purify the molecule of interest from a mixture (e.g., impurities), and then the purified molecule is subjected to MW determination by a mass analyzer. Heterogeneous glycoforms of mAb, separated by CE or HPLC are identified through their MWs by MS. SEC-light scattering, can be employed to measure the sizes and the molecular weights of proteins, including protein aggregates (Arakawa et al., BIOPROCESS INTERNATIONAL (2006) 4(10): 42-43; Arakawa et al., BIOPROCESS INTERNATIONAL (2007) 36-47).

2. Protein Primary Structure

The primary structure of protein refers to the amino acid sequence of the polypeptide. It is very important to confirm the amino acid sequence of the protein backbone since amino acid modifications may occur during manufacture or storage of biologics, which can result in the loss of stability and/or biological function. A peptide profile of amino acid sequence on proteins including any amino acid modifications is one of major quality characteristics for protein biologics. Sequencing can be accomplished using a variety of approaches.

Edman degradation is a traditional method used in peptide sequencing (Schroeder, METHODS ENZYMOL (1967) 11:445-461; Niall, METHODS ENZYMOL. (1973) 27:942-1010). Chemical reagents such as phenylisothiocyanate (PITC) can couple with the N-terminal amino group of a protein or peptide to form a phenylthiocarbamyl (PTC) adduct which can be cleaved under acidic conditions. Due to the limitation of Edman degradation reactions, which have to proceed from the N-terminal of peptides, this method cannot be used to sequence peptides on protein where N-terminal amino acid is modified. Thus, mass spectrometry is the preferred method for peptide sequencing.

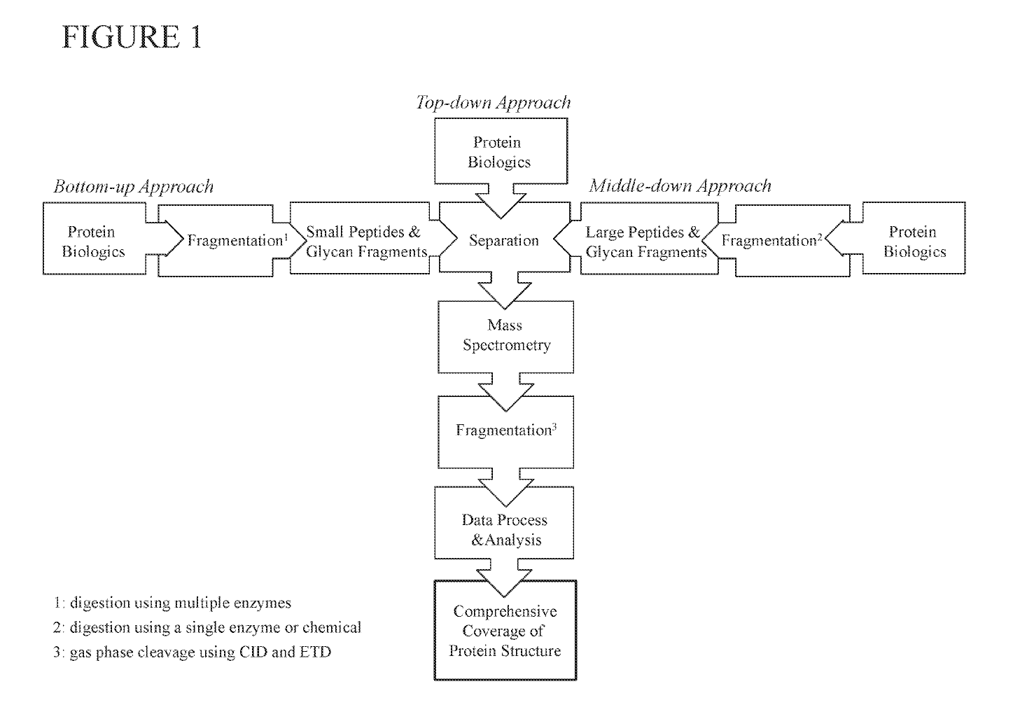

Mass spectrometry-based platforms (e.g., bottom-up, middle-down, and top-down) using multiple fragmentation techniques can be used to obtain a comprehensive coverage of peptide sequence on protein biologics (see FIG. 1). In the bottom-up approach, which serves as a standard method for peptide sequencing, a protein is digested into small peptide fragments (Wysocki (2004) supra; Scigelova (2006) supra; Wu et at., JOURNAL OF PROTEOME RESEARCH (2007) 6: 4230-4244). These small peptide fragments then are identified by MS/MS typically using CID. Nevertheless, the whole protein sequence can still be uncertain, mainly because of redundant peptide sequences present in the protein or loss of labile post-translational modifications. The middle-down approach is an intermediate method to the bottom-up approach (Wu et al. NAT. METHODS (2013) 9(8): 822-824). A protein is digested into large peptide fragments, which are subsequently fragmented using CID and/or ETD, on a mass spectrometer. In general, use of bottom-up and middle-down approaches covers most of peptide sequences on proteins. Yet, a full coverage of protein sequence may not be possible for some proteins. A top-down approach can be used as an alternative to bottom-up and middle-down methods (Wysocki (2004) supra; Scigelova (2006) supra). An intact protein without digestion can be directly measured by mass analyzers and subsequently fragmented by CID, HCD, and ETD. Top-down sequencing allows locating post-translational modifications and differentiating isomers which could be lost in bottom-up and/or middle-down approaches. The use of two or more of these approaches of mapping peptide fragments enables high probabilities of a full coverage of peptide sequences on proteins.

For bottom-up sequencing, a protein is subjected to digestion using a single enzyme such as trypsin and multiple enzymes such as (trypsin and Lys-C), (trypsin and Asp-N), or (trypsin, Lys-C, and Asp-N), etc. to generate many small peptide fragments in solution (see the section of sample preparation for details). Then the digested protein containing a mixture of small peptide fragments is subjected to HPLC separation and subsequent MS/MS analysis.

Two types of HPLC separation can be performed depending on the sensitivity of peptides. If sensitivity is not a concern, the digested protein (for example, 10 μg) is subjected to a reverse-phase LC separation using a C18 column (for example, Agilent 300SB C18 column, 2.1 mm id×15 cm). Normally, mobile phase A contains 0.1% formic acid in water and mobile phase B consists of 0.1% formic acid in acetonitrile. The separation of peptides is achieved through a gradient (for example, from 2% mobile phase B to 40% B in 60 minutes at a flow rate of 200 μL/min) using a HPLC pump (for example, Agilent 1200 HPLC system or Dionex UltiMate 3000 RS pump) coupled with a mass spectrometer. Followed by HPLC separation, peptides are introduced into a mass analyzer through an electrospray interface under a positive or a negative ionization mode. If sensitivity becomes an issue due to the limited protein source or a poor recovery of peptides digested from a protein. HPLC separation can be carried out using a nano-capillary C18 column (for example, Michrom Bioresources Magic C18 or Thermo Acclaim PepMap RSLC C18 column, 100 Å pore, 2 μm, 75 μm id×15 cm) on a nano-capillary HPLC pump (for example, Dionex UltiMate 3000 RSLCnano pump). The separation of peptides on a nano-capillary column is accomplished using a gradient (for example, from 2% to 60% B in 90 minutes) at a flow rate of 200 nL/min. A nano-spray ion source (for example, Thermo Nanospray Flex) is equipped with a mass spectrometer (LTQ obitrap Elite ETD) to introduce the sample after nano-capillary HPCL separation.

Once peptides are delivered into a mass spectrometer, peptides are positively charged (known as precursor ions) and subsequently fragmented by applying CID energy to produce smaller fragments (known as product ions). For example, LTQ Obitrap Elite ETD can be operated under a data-dependent mode to allow automatically switch between MS, CID-MS², ETD-MS², and/or HCD-MS². Mostly, MS, CID-MS², and ETD-MS² are sufficient for peptide sequencing. CID on an isolated charge-reduced species (i.e., CRCID) generated front ETD-MS² is normally used to characterize phosphorylation, disulfide bond formation, and glycosylation. After a survey full-scan MS spectrum (for example, from m/z 400 to 2000), subsequent CM-MS² and ETD-MS² activation scan steps can be performed on the same precursor ion (for example, a peptide fragment generated in solution) over the same m/z scan range as that used for the full-scan MS spectrum. The precursor ion is isolated using the data-dependent acquisition mode with a ±2.5 m/z isolation width to select automatically and sequentially a specific ion from the survey scan. Then, an additional CID-MS³ step is performed on an isolated precursor ion with a ±5 m/z isolation width and with the highest intensity from the CID-MS², ETD-MS², or HCD-MS² scan. CID-MS², ETD-MS², and HCD-MS² can be repeated in sequence to select for fragmentation of subsequent highest intensity precursor ions from the first survey scan. The peptide sequence can be identified by assembling various types of fragment ions, for example, b and y ions mainly produced by CID-MS²; c and z ions generated by ETD-MS² for each peptide with assistance of software (see assignment of peptide sequence for details) (Steen et al., MOLECULAR CELL BIOLOGY (2004) 5:699-711).

In the middle-down approach, the protein is subjected to digestion using a protease (for example, Lys-C, Asp-N, or Glu-C) or a chemical (for example, CNBr) to generate large peptide fragments in solution. Then the digested protein containing a mixture of large peptide fragments is subjected to HPLC separation and subsequent MS/MS analysis.

In the top-down approach, the intact protein is subjected to nano-capillary LC MS/MS analysis using multiple fragmentation techniques (for example, CID, FICD, and ETD). The fragmentation spectra generated from an intact protein are more complicated, compared to the bottom-up and the middle-down approaches. To avoid collecting muddled data from a mixture of proteins, the protein of interest usually needs to be purified or fractionated (for example. GELFREE™ fractionation; Tran et al., ANALYTICAL CHEMISTRY (2008) 80(5): 1568-1573) prior to MS analysis. After purification or fractionation, the protein sample is separated using a nano-capillary HPLC column (e.g., 1000 Å, 5 μm, polymer reversed phase PLRP-S, 75 μm id×10 cm). CID, HCD, and ETD fragmentation are applied to the most intense charge state of an intact protein on the Thermo LTQ Obitrap Elite ETD mass spectrometer.

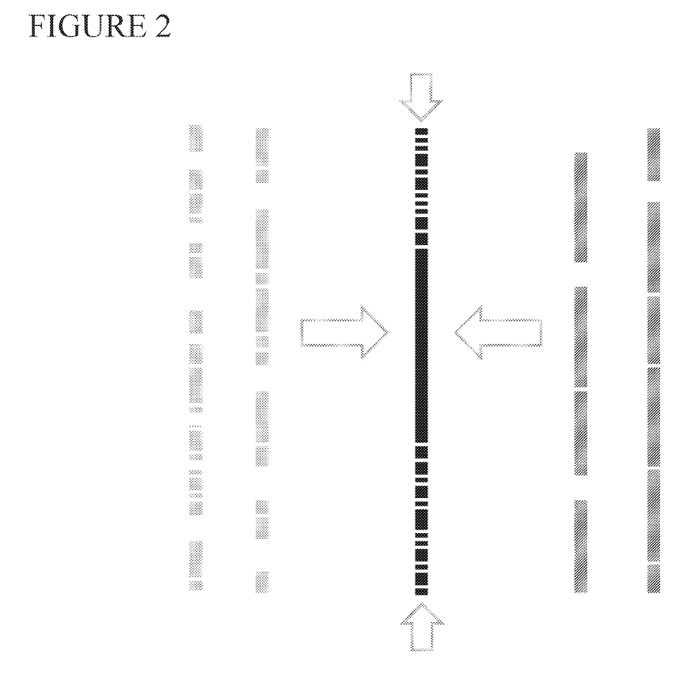

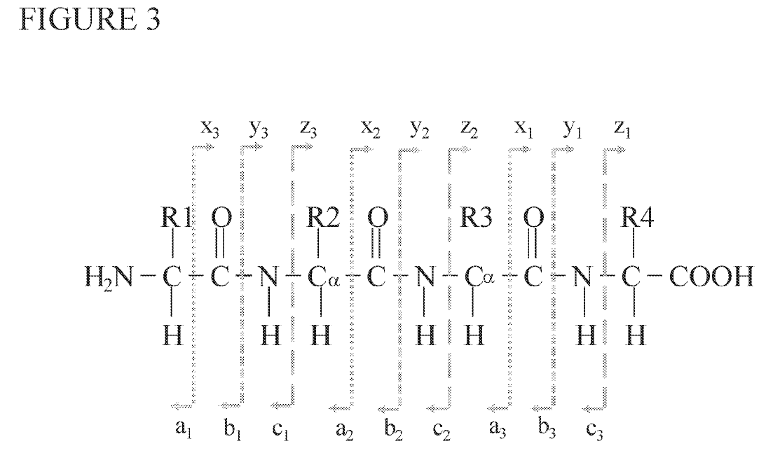

The protein or peptide sequence can be created using a variety of approaches that assign the peptide sequences. A staggered strategy of mapping fragments obtained from bottom-up, middle-down, and/or top-down methods, as shown in FIG. 2, is used to confirm the protein sequence. Based on the fragmentation pattern (for example, a, b, c, x, y, z ions, in FIG. 3) observed from MS/MS spectra, the sequence of each peptide fragment can be assigned using software(s) to obtain theoretical digested peptide masses to match with the experimental data (i.e., MS/MS fragmentation spectra). For example, MS/MS spectra (bottom-up and middle-down sequencing), generated on a LTQ Obitrap Elite ETD and a Q Exactive Plus mass spectrometers are processed using Thermo PepFinder software that has the predicted MS/MS algorithm incorporated to assign fragmentation spectra to the most probable peptide sequence. The spectra generated in the CID-MS², ETD-MS², HCD-MS² and/or CID-MS³ are searched against spectra of theoretical fragmentations (for example, b and y ions) using Pep-Finder to assign peptide identification based on the mass accuracy, the similarity of MS/MS pattern, status of disulfide bond (reduced or non-reduced), N-glycosylation (CHO N-glycan, human N-glycan, or none), mass changes for unspecified modifications, and statistical confidence (for example, 90%) etc. Final confirmation of the most probable peptide sequence assignments is obtained by inspection of individual mass spectra with the preferred fragmentation patterns in the observed CID-MS², ETD-MS²′HCD-MS², and/or CTD-MS³ spectra. The peptide assignment for top-down MS/MS spectra can be performed using Mascot (Matrix Science) or ProSight Suite (Thermo Fisher Scientific).

3. Amino Acid Modifications

Amino acid modifications can occur to biological molecules during manufacture, formulation, or storage as a consequence of protein degradation or post-translational modifications. Protein degradation can occur as a result of chemical and physical modification. Chemical modification can change peptide backbone amino acids through oxidation, deamidation, isomerization, and racemization. Physical modification can trigger unfolding, misfolding or aggregation on proteins. Post-translational modifications usually happen during production of biologics in a cell or a cell system. Common post-translational modifications include acetylation, methylation, phosphorylation, and glycosylation, which may take place in N-terminal, C-terminal, or side chain of amino acid residues. The altered or modified amino acids can be detected via peptide sequencing using enzymes, chemicals, and MS fragmentation techniques, as described above. The key for identifying amino acid modifications is to locate modification sites which can be found according to mass differences between modified (observed MS spectra) and un-modified (theoretically predicted MS spectra) amino acids. To confirm the modification sites, the peptide containing modified amino acid(s) is subjected to MS/MS analysis. Use of MS/MS methods depends upon the modification site of interest. Examples of characterization of amino acid modifications are illustrated below.

### a. N-Terminal Modifications

N-terminal modifications include, but not limited to, acetylation, methylation, formulation, cyclization of glutamine, myristoylation, phosphorylation, and glycosylation, etc. (Meinnel et al., PROTEOMICS (2008) 8: 626-649). For example, acetylation takes place mostly at a lysine (Lys) residue; formylation is often observed on an N-methionine (Met) residue; cyclization converts glutamine (Gln) to pyroglutamic acid (pGlu) which is observed in mAb; myristoylation usually occurs M a glycine (Gly) residue. Based on the type of enzyme chosen for digestion, the digested protein containing peptide fragments including N-terminal peptide is subjected to LC-MS/MS analysis. The mass of the N-terminal peptide with a modification on amino acid can be monitored by LC-MS on a mass spectrometer. The type of N-terminal modification can be distinguished by mass differences (for example, +42 for acetylation; +14 for methylation; +28 for formylation; +17 for cyclization of glutamine to pyroglutamic acid; +210 for myristoylation.; +80 for phosphorylation) through the MS full scan followed by LC separation. Once detecting the predicted mass of the modified N-peptide, the N-peptide containing amino acid modification is selected as a precursor ion and then subjected to CID-MS² and/or ETD-MS² to produce more fragments. Generally, the modification on the N-amino acid residue can be detected by additional masses on b fragmentation ions observed from CID-MS² (for example, +42 for acetylation) and no mass shift on y fragmentation ions.

### b. C-Terminal Modifications

C-terminal heterogeneities often occur in recombinant monoclonal antibodies (Liu et al., JOURNAL OF PHARMACEUTICAL SCIENCES (2008) 97(7): 2426-2447). One of the most common C-terminal heterogeneities (Liu (2008) supra) is the incomplete C-terminal lysine processing of the heavy chain during production of monoclonal antibodies to produce three antibody species containing zero, one, and two C-lysine residues. To characterize C-terminal lysine modification, the antibody is subjected to digestion using enzymes and then separation of heavy chains from light chains using molecular weight cut-off centrifugation filters (for example, 10,000 Da cut-off). The heavy-chain fragments containing C-terminal processing lysine peptide species are subjected to LC separation (e.g., use of reverse-phase LC column), followed by MS/MS analysis. Generally, peptides containing heterogeneous C-terminal lysine residues can be separated by LC and then identified by MS. For example, a reduction of 128 Da in mass indicates a removal of one C-terminal lysine residue. The positive charge state on the removal of one C-terminal lysine peptide is decreased by 1 unit as well. Therefore, various C-terminal lysine species can be identified by LC-MS based on the charged state and the masses. C-terminal amidation species are also often noted in the heavy chain of monoclonal antibodies (Tsubaki et al., INTERNATIONAL JOURNAL OF BIOLOGICAL MACROMOLECULES (2013) 52: 139-147). Like C-terminal lysine modification, amidation (a reduction of 1 Da) occurs as a result of post-translational modification. Peptidylglycine α-amidating monooxygenase (PAM) can cleave C-terminal glycine (Gly) and amidate the penultimate amino acid, resulting in a reduction of 58 Da in mass if the second last amino acid to Gly residue is proline (Pro) or leucine (Leu). Similar to C-terminal lysine species, the heavy-chain peptide fragments are analyzed by MS using CID-MS² followed by LC separation, C-terminal amidation species can be identified.

### c. Oxidation

Biologics can be oxidized if oxygen radicals or metals are present in the environment. In proteins, the most common oxidation occurs to amino acids containing a sulfur atom such as methionine (Met) and cysteine (Cys) or an aromatic ring such as histidine (His), tyrosine (Tyr), tryptophan (Trp), and phenylalanine (Phe) (Patal et al., BIOPROCESS INTERNATIONAL (2011) 20-31). For example, the sulfur atom (S) on Met reacts with oxygen radicals in solution to form methionine sulfoxide (S═O) and methionine sulfone (O═S═O). Cys oxidation opens disulfide links and forms new disulfide bonds, leading to mispaired disulfide bonds and scrambled disulfide bridges. Spontaneous oxidation (also known as auto-oxidation) may cause Cys to form sulfinic acid (SOOH) and cysteic acid (SOOOH) if metal ions are present in solution. His oxidation occurs through the reaction between imidazole rings to generate oxidation products such as 2-oxo-histidine (2-O-His), aspartic acid (Asp), and asparagine (Asn). Trp can be oxidized by light (also known as photo-oxidation) to form oxidation products such as N-formylkynurenine and kynurenine (Li et al., BIOTECHNOLOGY AND BIOENGINEERING (1995) 48: 490-500). Photo-oxidation of Tyr can form 3,4-dihydroxyphenylalanine (DOPA) and dityrosine, resulting in covalent aggregation through forming Tyr-Tyr cross links. Protein oxidation can be measured through LC-MS analysis of a protein digest. Use of theoretically predicted masses of peptides containing potential oxidation products (for example, +16 Da or +32 Da) and fragmentation pattern observed on peptide fragments can identify oxidation products on protein.

### d. Deamidation, Isomerization, and Racemization

Deamidation occurs to many recombinant proteins by removing an amide group from an amino acid such as asparagine (Asn) and glutamine (Gln) (Patal (2011) supra). Deamidation is a non-enzymatic process that can take place spontaneously on proteins or peptides in vivo or in vitro systems. Consequently, proteins undergo isomerization and racemization after deamidation. For example, Asn is initially converted to aspartic acid (Asp) by the non-enzymatic deamidation process, which can be identified through a mass shift of +1 Da on a mass spectrometer. Isoaspartic acid (isoAsp), as the most commonly found deamidation product, is then formed via isomerization of Asp. The isoAsp and Asp peptide products are normally separated by LC and subsequently identified by MS/MS. Besides, succinimide intermediate generated during Asn deamidation process can be converted to D-Asp (refer to racemization). Overall, the rate of deamidation on an intact protein is very slow; whereas the deamidation rate can be increased significantly for peptides under alkaline condition (Hao et al., (2011) MOLECULAR & CELLULAR PROTEOMICS 10.10). For example, D,L-Asp and D,L-isoAsp peptides (predominated with isoAsp peptides) are formed as a consequence of deamidation, isomerization, and racemization during trypsin digestion using buffer at pH 8. Normally, deamidation of Gln is much slower compared to the deamidation of Asn.

It is important to avoid inducing in-vitro deamidation during sample preparation while identifying in-vivo deamidation sites on proteins. Modified sample preparation procedures may be needed to identify deamidation modifications on proteins. For example, a protein sample can be subjected to trypsin digestions under pH 6.5 and pH 8, respectively. In-vivo deamidation products can be distinguished from in-vitro products by profiling the digested proteins (pH 6.5 vs. pH 8) by LC-MS. Peptides obtained from protein digestion at pH 6.5 serves as a control to filter in-vitro induced deamidation peptide products at pH 8.

4. Post-Translational Modifications

Post-translational modifications play an essential role in protein functions which regulate cellular process. Post-translational modifications occur after the translation of mRNA. It is a biochemical process where amino acid residues are covalently modified by removing or adding molecules in a protein. These modifications can change a protein's folding, biological function, immunogenicity, and/or stability (Farley et al, METHODS IN ENZYMOLOGY (2009) 463: 725-762, Walsh et al., NATURE BIOTECHNOLOGY (2006)24(10): 1241-1252). Post-translational modifications include, but are not limited to, acetylation, acylation, γ-carboxylation, β-hydroxylation, disulfide bond formation, glycosylation, methylation, phosphorylation, proteolysis processing, and sulfation. Among these modifications, acetylation, methylation, amidation, phosphorylation, and glycosylation are commonly found in approved therapeutic protein drugs and candidates in discovery or clinical trial stages. Heterogeneous species can be formed after post-translational modifications, such as glycosylation or amidation, which may or may not alter protein folding and function. Thus, characterization of post-translational modifications provides structural insight to enable associating structure with biologic functions. Use of mass spectrometric methods for characterization of post-translational modifications on proteins, MS fragmentation techniques play critical roles in producing specific types of fragments to allow identifying post-translational modification sites in Which amino acid residues are modified. Structural elucidation of amidation is described above in the section “C-Terminal Modification”. Characterization of glycosylation is illustrated in the following section “Higher-Order Structures”. Examples of characterization of proteins with methylation, acetylation, and phosphorylation modifications are demonstrated, respectively below.

### a. Methylation and Acetylation

Methylation involves adding one or more methyl groups onto amino acids. For example, N-methylation can be found at the N-Terminal alanine, isoleucine, leucine, methionine, phenylalanine, proline, tyrosine and/or the side chains of lysine, arginine, glutamine, asparagine or the imidazole ring of histidine residues (Paik et al., YONSEI MEDICAL JOURNAL (1986) 27(3): 159-177). O-methylation can be observed either at a C-Terminal cysteine, leucine, lysine or at the side chain of glutamic acid and aspartic acid residues (Paik (1986) supra). S-methylation can be noted at the side chains of methionine and/or cysteine residues. Acetylation transfers an acetyl group to the side chain of lysine (also known as lysine acetylation) or the N-terminal amino acid residue (also known as N-terminal acetylation). in general, methylation and acetylation modifications on proteins remain unchanged during sample preparation (for example, digestion by enzymes). After protein digestion, peptide fragments in solution including methylated and/or acetylated peptides are subjected to LC-MS analysis. Methylated peptide species can be identified by additional masses (for example, +14 Da for mono-methylation, +28 Da for di-methylation) followed by LC separation. Though tri-methylation and acetylation modifications provide the same additional mass of 42 (Da), the identification can be carried out by the CID-MS² fragmentation. For example, an unique immonium ion of m/z 126 can be observed in acetylated lysine but not present in tri-methylated lysine residues (Farley (2009) supra). In addition, a neutral loss of 59 Da, corresponding to the loss of trimethylamine, is unique for a tri-methylated lysine.

### b. Phosphorylation

Phosphorylation typically occurs at serine, threonine or tyrosine residues of proteins or peptides. In general, phosphorylation is a reversible post-translational modification occurring in cellular process to control protein activities. Phosphorylation is one of liable post-translational modifications. The phosphate groups on serine and threonine residues can compete with the peptide bones as preferable cleaved sites. Upon CID activation, peptides containing phosphorylated amino acid residues tend to lose the phosphor groups before they fragment along with the peptide backbone. As a result, mixed fragments are obtained, which cannot be differentiated between unmodified and phosphorylated peptides. To avoid obtaining ambiguous peptide sequences from phosphorylated proteins, a combination of CID (CID-MS²), ETD (ETD-MS²) and CRCID (CID-MS³) fragmentation techniques can be used to characterize phosphorylation modifications on proteins (Wu et at., JOURNAL OF PROTEOME RESEARCH (2007) 6: 4230-4244).

For example, after denaturation, reduction, and alkylation a phosphorylated protein sample is subjected to digestion using Lys-C to generate large proteolytic peptides. A large pore size of monolithic LC column such as polystyrene-divinylbenzene (PS-DVB, 50 μm i.d×10 cm) can be used to separate large peptides including unmodified and phosphorylated peptides. CID and ETD are operated under both dependent and independent modes on a mass spectrometer (for example, Thermo LTQ Obitrap Elite ETD). Under an independent mode, use of CID and ETD can manually select less intensity of precursor ions (usually phosphorylated peptides) for subsequent fragmentation (for example CID-MS³), which is normally missed during data dependent experiments. Furthermore, fewer fragment ions (c and z ions) are obtained for a large peptide with a less charge (for example, +2) in the ETD-MS² scan, resulting in insufficient fragmentation for peptide assignment. A combination of using ETD (ETD-MS²) following CRCID (CID-MS³) via isolating an product ion produced in the ETD scan step can produce substantial c and z ion series along with phosphorylation sites on peptides. The peptide assignment for phosphorylated peptides and unmodified peptides is achieved using software(s) (for example, PepFinder and/or Proteome Discoverer). Besides mapping fragment ions, HPLC retention times and masses (for example, a loss of 98 Da as signature of phosphorylated peptide) are the key to assign the peptide identity.

5. Higher Order Structures

Higher order structures (HOS) of a biologic protein include the secondary, tertiary, and quaternary structures. HOS provide a three-dimensional (3D) confirmation, which plays an important role in its biological function. HOS are considered to be critical quality attributes because changes in HOS may affect efficacy or safety of biologic drugs. Characterizing HOS of a biologic protein is required by regulatory agencies (for example, USFDA A quality by design, QbD and ICH Q5E (ICH HARMONISED TRIPARTITE GUIDELINE “Comparability of Biotechnological/Biological Products Subject to Changes in Their Manufacturing Process,” Q5E, Current Step 4 Version, dated Nov. 18, 2004)). HOS are often required during manufacturing of biologics (for example, comparability evaluations), formulation, stability assessment, and process development. Circular dichroism (CD) spectroscopy (Li et al., JOURNAL OF PHARMACEUTICAL SCIENCES (2011) 100(11): 4642-4654), X-ray crystallography (Harris et al., J. MOL. BIO. (1998) 275: 861-872), and nuclear magnetic resonance (NMR) (Amezcua et at., JOURNAL OF PHARMACEUTICAL SCIENCES (2013) 102(6): 1724-1733) are the conventional tools used to analyze HOS of a protein.

Hydrogen deuterium exchange coupled with mass spectrometry (HDX MS) can be used to probe HOS of a biologic. Unlike CD spectroscopy, FIDX MS can provide a 3D confirmation of an intact molecule (Engen, ANAL. CHEM. (2009) 81(19): 7870-7875) and a local confirmation of fragments of a biologic, such as peptides (for example, peptide epitopes) (Coates et al., RAPID COMM. MASS SPECTROM, (2009) 23: 639-647). For example, the exchange of protein backbone amide hydrogen with deuterium (CO-NH→CO-ND) provides conformational dynamics of the molecule in solution at a physiological pH (for example, pH 7.5, room temperature). After quenching a HD exchange reaction (for example, pH 2.5, 0° C.), a biologic protein is subjected to digestion (for example, pepsin digestion at pH 2.5) and digested peptides then are analyzed by MS. The HDX rate observed in peptides provides an indication of protein HOS, which can be measured based on the mass gain over time in solution via MS. For example, slow exchange occurs in regions buried in the core of a 3D structure and in heavily glycosylated peptides. Fast exchange often occurs in regions located on the surface of protein structure and in peptides with little or no glycosylation. Advantages of HDX MS over x-ray crystallography and NMR spectroscopy are: 1) that it provides dynamic conformational information of native biologics in solution; 2) that it is unlimited by the size of proteins or biologics being interrogated; and 3) sensitivity (i.e., less material required for HDX MS analysis) (Berkowitz a al., NATURE REVIEWS DRUG DISCOVERY (2012) 11: 527-540). HOS of biologics also include disulfide bonds, disulfide knots, and glycosylation, which are described in the following sections.

### a. Disulfide Bonds

Disulfide bonds (—S—S—) primarily control the folding of three-dimensional protein structure, and generally fall into three groups: 1) intra-chain disulfide bonds; 2) inter-chain disulfide bonds; and 3) disulfide knots. In general, intra-chain disulfide bonds stabilize the tertiary structure and the inter-chain disulfide bonds involve in stabilizing quaternary structure. Disulfide knots can improve protein structural stability. Any modifications to the process of producing a biologic (e.g., changes in cell lines, cell culture medium, agitation force etc.) have the potential to cause protein conformational changes due to disulfide bond rearrangements (for example, unpaired or mispaired disulfide bonds). Thus, disulfide bonds are critical structural attributes, which need to be monitored for quality control purposes during manufacture or storage of biologic or biologic reference.

Intra-chain disulfide bonds occur within a single polypeptide whereas inter-chain disulfide bonds are formed between two polypeptide chains through oxidation of thio (—SH) groups on cysteine residues. A conventional approach for characterizing disulfide bonds includes comparing reduced and non-reduced peptide profiles to help locate disulfide bonds on peptide backbones. A protein sample is digested by enzyme with and without reduction and alkylation to generate two protein digests (for example, protein digest 1 (PD1) with reduction and alkylation; protein digest 2 (PD2) without reduction and alkylation). PD1 and PD2 are subjected to LC-MS analysis. The disulfide-linking peptide (DSLP) can be found in the PD2 sample using a theoretical mass to locate retention time on LC-MS mass chromatogram. Then, the sequence of DSLP can be determined using ETD to cleave a disulfide bridge followed by CID to break peptide amide bonds subsequently. As expected, DSLP should not be detected in a PD1 sample. However, LC-MS analysis cannot differentiate an intra-chain disulfide bond from an inter-chain disulfide bond. The sample can be subjected to SDS-PAGE gel electrophoresis under reduction and non-reduction conditions. For a sample containing an intra-chain disulfide bond, no additional band can be discerned in both reducing and non-reducing gels. If it is an inter-chain disulfide bond, two additional bands (lower molecular weights) can be found in a SDS-PAGE gel run under reducing, conditions.

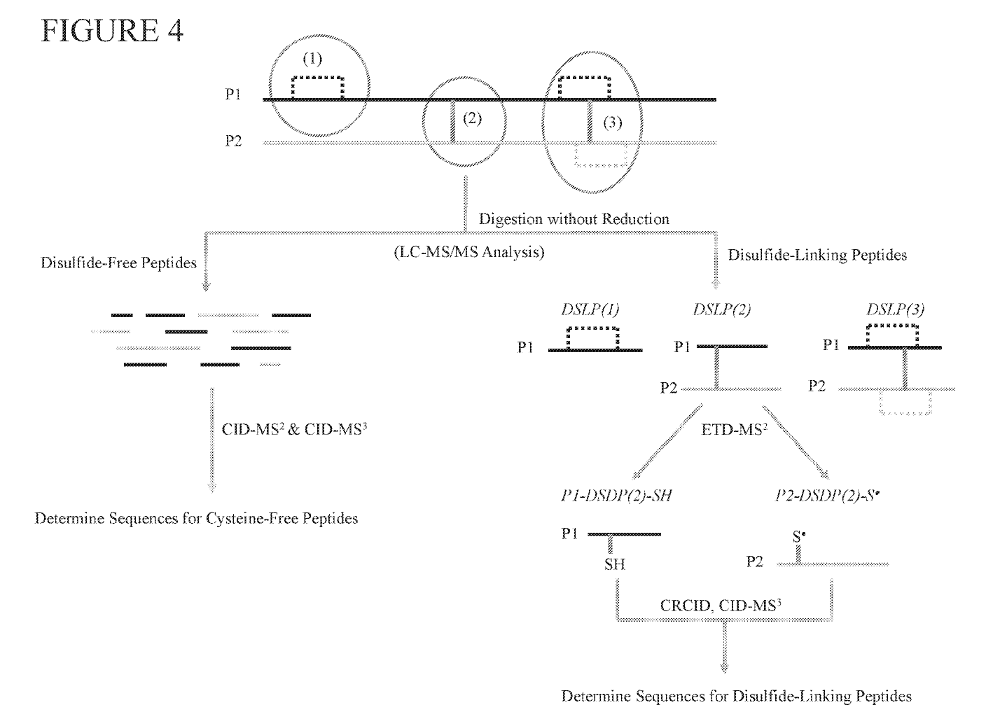

If a molecule of interest contains multiple disulfide bonds, the analysis of disulfide bonds is more complicated. Depending on the protein sequence and the locations of disulfide bonds, a strategy of using multiple enzymes and multi-fragmentation techniques to digest proteins into peptides containing only a single disulfide bond is ideal for mapping the disulfide bonds (Wu et al., ANAL. CHEM. (2009) 81(1):112-122); Wu et al., ANAL. CHEM. (2010) 82(12): 5296-5303). By way of example, and as shown in FIG. 4, an exemplary protein consisting of two polypeptides (P1 and P2) connected via two inter-chain disulfide bonds, where two intra-chain disulfide bonds are located in Pi and one intra-chain disulfide bond is placed in P2 can be characterized as follows. The protein without reduction is digested using multiple enzymes to produce 3 disulfide-linking peptides, referred to as DSLP(1), DSLP(2), DSLP(3), and many disulfide-free peptide fragments. Then, this protein digest (without reduction) is subjected to LC-MS/MS analysis through a reverse-phase HPLC separation (for example, use of Agilent Zorbax 300SB-C18 column, 5 μm particle size, 2.1 mm id×15 cm) coupled with a mass spectrometer (LTO Obitrap Elite ETD). Again, theoretical masses of three predicted DSLPs are monitored to find their corresponding LC-MS chromatograms. Once the three DSLPs are located, the disulfide bonds can be cleaved using ETD (ETD-MS²) on DSLPs to produce disulfide dissociated peptides (DSDPs). For example, DSLP(2) is broken into two disulfide dissociated peptide species: P 1-DSDP(2)-SH and P2-DSDP(2)-S* (see FIG. 4), which are further fragmented to yield c and z ions using CRCID followed by ETD-MS², and to produce b and y ions using CID-MS³ followed by CID-MS². The disulfide-free peptides are fragmented by CID-MS² and/or CID-MS³.

The assignment of DSLPs is based on the assumption that the locations of disulfide bonds on the polypeptide backbones (P1 and P2) are predictive of the sequences for DSDPs. Hence, The fragmentation spectra of PI-DSDP(2)-SH and P2-DSDP(2)-S* including CID-induced b and y ions and ETD-induced c and z ions are used to search against theoretical fragmentations of this given protein using software(s) such as PepFinder and Proteome Discoverer. The sequences of disulfide-free peptides are assigned in a similar manner.

To assure the assignment of DSLPs (for example, P1 or P2 peptide fragments), the protein sample is subjected to a SDS-PAGE gel electrophoresis under reducing conditions. Two separated gel bands corresponding to polypeptides P1 and P2, respectively, are cut from the gel and digested with multiple enzymes. The extracted peptides from each gel piece are analyzed by LC-MS/MS. The sequences of polypeptides P1 and P2 then are determined using CID-MS² and/or CID-MS³.

If predicted DSLPs cannot be found, disulfide bond rearrangement may have occurred in the protein. To verify the absence of predicted DSLPs, the protein can be digested using the same multiple enzymes with reduction and subjected to peptide sequencing by LC-MS/MS methods as described above. Through peptide mapping and sequencing, the cysteine residues can be located on the peptide backbones. Then, the protein can be subjected to digestion using different multiple enzymes with and without reduction. This assumes that the disulfide bridges are shuffled to bear unexpected DSLPs such as unpaired or mispaired DSLPs. Thus, the use of different multiple enzymes can recover re-arranged DSLP fragments. These un-paired or mispaired. DSLPs can be characterized and identified by LC-MS methods as described above.

### b. Disulfide Knots

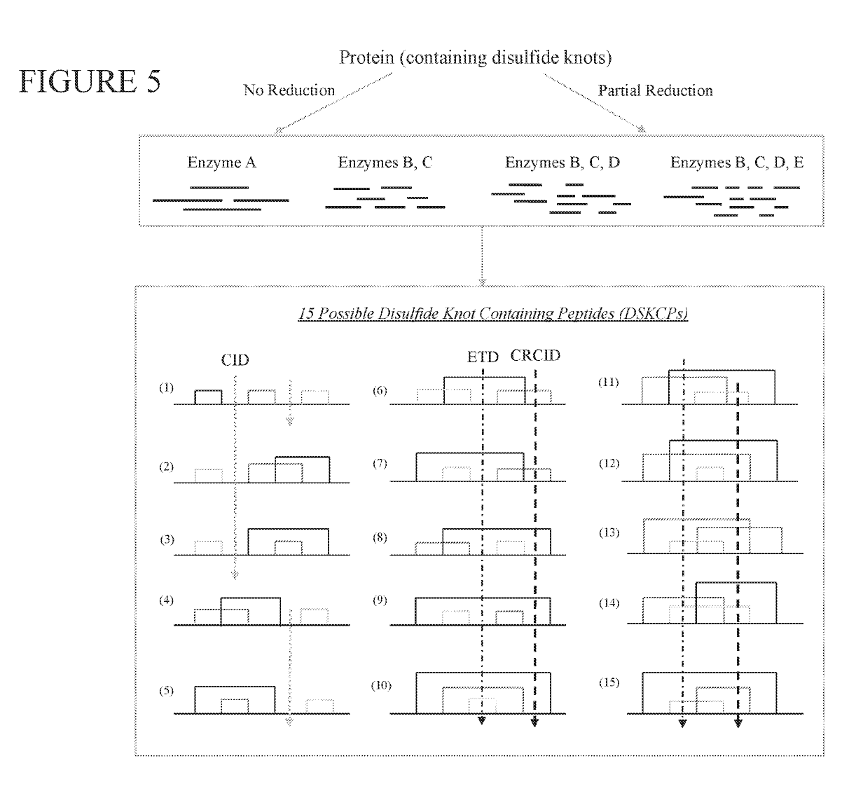

Disulfide knots are structural motifs often found in proteins and typically comprise at least three disulfide bonds (six cysteine residues), where one disulfide bond passes through the ring of the other two disulfide bonds. Some therapeutic protein biologics (for example, recombinant human arylsulfatase A) contain disulfide knots, which can be scrambled or shuffled during expression, purification, or storage. It can be difficult to verify a protein bearing disulfide knots with a correct position since there are many ways to arrange a disulfide knot (Ni et al., J. AM. SOC. MASS SPECTROM. (2012) 24: 125-133). Enzymes or CID typically do not cut the peptide backbone disposed within a disulfide knot. The generation of desirable sizes of peptide fragments is important in the successful characterization of biologics containing disulfide knots. Thus, a process of using staggered multi-enzymes and multiple fragmentation techniques has been developed, as is shown schematically in FIG. 5.

As shown in FIG. 5, a sample is subjected to digestion without reduction and/or with partial reduction using a series of staggered multiple enzymes. Enzyme A can be a single enzyme such as pepsin to produce fragments under an acidic condition. It is known that disulfide scrambling can take place under alkaline conditions. Thus pepsin digestion (pH 2) can avoid disulfide shuffling and serve as a negative control for scrambled disulfide bonds. Nevertheless, limited or large peptide fragments are produced by pepsin digestion due to the presence of disulfide knots, Enzymes B and C (for example, trypsin and Lys-C), B, C, D (for example, trypsin, Lys-C, Asp-N), and possibly B, C, D, and E (for example, trypsin, Lys-C, Asp-C, and PNGase F if there is glycosylation on a protein) can yield different, small sizes of fragments. The multiple enzyme digestions can be carried out at pH 6.8 and pH 8 to monitor false disulfide knots if induced during sample process. Yet, kinetically favorable mismatched disulfide bridge(s) can be formed from two adjacent cysteines during enzymatic digestion. To avoid artifacts associated with the breakage of cysteine knots introduced during sample processing, a method of partial reduction for example, a protein sample reduced by TCEP in 6M guanidine hydrochloride/sodium acetate buffer, pH 4.6, at 37° C. for no more than 20 minutes) and alkylation (for example, a partially reduced protein sample incubated with NEM at 37° C. for 60 minutes in the dark) can be used to block the formation of S—S bonds from two adjacent cysteines prior to the enzymatic digestion (see FIG. 5). Then, all the digested samples are analyzed by LC-MS/MS using CID (CID-MS), ETD (ETD-MS²), and CRCID (CID-MS³) to generate disulfide free fragments and disulfide knot-containing fragments, as shown in FIG. 5.

Although the use of theoretical masses can locate disulfide knot containing fragments on a LC-MS chromatogram, it can be difficult to profile fragmentation spectra to confirm the correct sequence defining a disulfide knot from multiple possible structural arrangements (for example, the 15 possible arrangements shown in FIG. 5). Use of multi-tier fragmentation techniques (CID→ETD→ETD/CID-MS³) can simplify the structural assignment for disulfide knot containing peptides (Ni (2012) supra). For example, to identify a correct disulfide knot position from 15 possible structural arrangements having the same precursor mass shown in FIG. 5, CID (dotted line) is used as the first tier to exclude the other ten possible structure rearrangements (for example, DSKCPs 6-15, FIG. 5). Only dissociated disulfide containing peptides can be observed for DSKCPs (1-5) using CID alone (FIG. 5). ETD (dash-dot line) serves as the second tier to fragment DSKCPs 6-15 if no dissociated disulfide containing peptides are observed after CID fragmentation in the first step. During this ETD step, disulfide knots are opened via cleavage of S—S bonds as illustrated in DSKCPs 6-15 (FIG. 5). Then, the use of CID-MS³ followed by ETD (i.e., CRCID; dashed lines) generates more fragment ions for the assignment of DSKCPs. DSCKP fragments obtained from protein digests without reduction (for example, pepsin, pH 2; trypsin, 6.5 and pH 8) and/or with partial reduction (for example, TCEP/NEM, pH 4.6) prior to digestion are carried out to assure the assignment of cysteine knots reflected from a native biologic.

### c. Glycosylation

Glycosylation is also important for the production of biologics because, for example, more than 90% of the protein drugs such as monoclonal antibodies are glycoproteins. Furthermore, glycosylation is the most complex post-translational modification, where sugar moieties play roles in protein binding, conformation, stability, and activity (Walsh (2006) supra). Glycosylation can significantly impact on the potency, pharmacokinetic, or immunogenicity of a biologic drug if any modifications (for example, changing cell lines) occur during the manufacturing process. Additionally, it can be difficult and Unpractical to produce a homogeneously glycosylated protein. Although the production of biologics is monitored under a good manufacturing process (GMP), heterogeneous species of glycoproteins (for example, different forms of glycan linked with a protein) can only be minimized. Thus, glycosylation is a critical attribute for a therapeutic protein.

Based on the glycosidic linkage between protein and glycan, glycosylation can be grouped into five types: N-linked (glycan attached to the amino group of asparagine), O-linked (glycan bound to the hydroxyl group of senile or threonine). C-linked (glycan added onto the indole ring of tryptophan), phospho-linked (glycol) linked to serine through phosphodiester bond), and glypiation (glycosylphosphatidylinositol anchor linked a phospholipid and a protein through a glycan (Ni (2012) supra). Among these five glycosylation types, the most common types are N-linked and O-linked. Characterization of glycosylation involves four steps: 1) glycan removal (known as deglycosylation); 2) glycosylation site determination; 3) peptide sequencing; and 4) glycol) analysis. Deglycosylation is essential for identification of the peptide and the site of glycosylation. After producing the peptide backbone via deglycosylation, glycan attached on the peptide (known as glycopeptide) can be predicted by subtracting the molecular weight of the peptide from that of the glycopeptide.

Based on the type of glycosylation, various approaches can be used to remove glycans from a glycoprotein. For example, PNGase F can remove most N-linked glycans except for a fucose-α(1-3) bound to the Asn-GlcNAc linkage. N-glycosidase A can be used to release oligosaccharides containing an all-3) fucose core. There is no enzyme like PNGase F that can remove “intact” O-linked glycans. Rather, the removal of O-linked glycans can be achieved using a series of exoglycosidases to hydrolyze various types of monosacchrides until only the Gal-β(1,3)-GalNAc core remains. O-glycosidase (endo-α-N-acetylgalactosamindase) can then release the Gal-β(1,3)-GalNAc core structure from the serine or threonine residues (Iwase et al., METHODS IN MOLECULAR BIOLOGY (1993) 14: 151-159). Determination of glycosylation site can be accomplished in parallel with peptide sequencing because N-linked asparagine or O-linked serine/threonine residues are the known as glycosylation sites. For glycan analysis, glycan can be collected after enzymatic or chemical digestion. The methods for characterization of N-linked and O-linked glycosylation are described below.

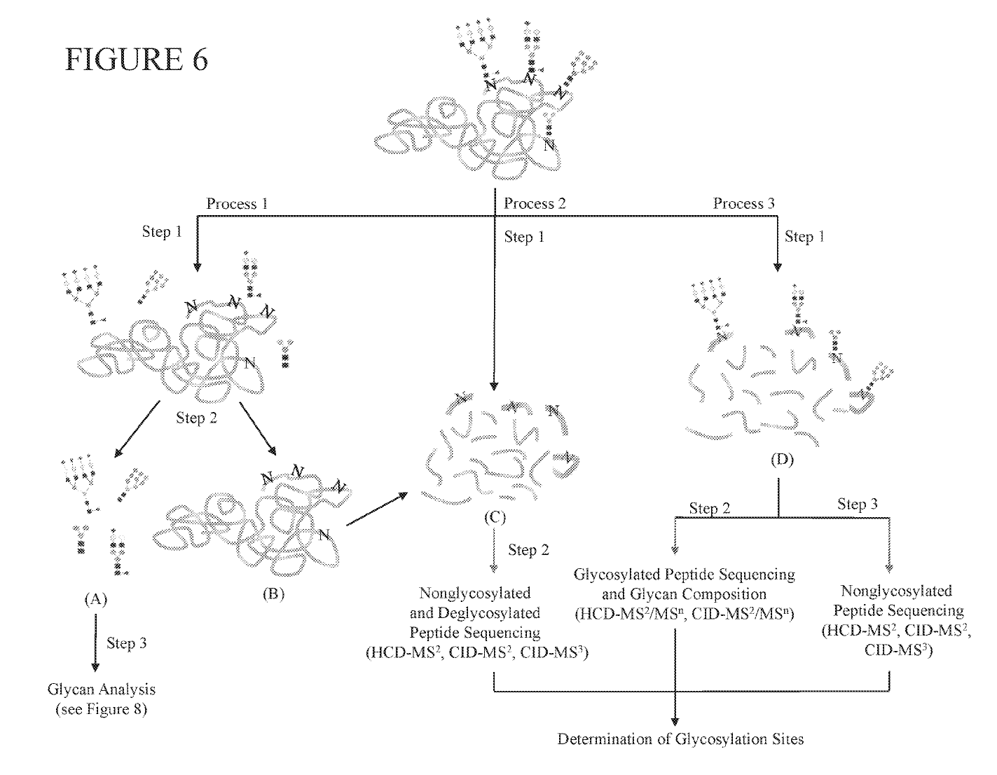

As shown in FIG. 6, N-linked glycosylation can be determined via three integrated processes: 1) deglycosylation (process 1); 2) multi-enzymatic digestion including deglycosylation (process 2); and 3) multi-enzymatic digestion without deglycosylation (process 3). For example, a glycoprotein sample is subjected to denaturation (6M guanidine hydrochloride, room temperature), reduction (200 mM DTT at 37° C. for 30 min), and alkylation (200 mM IAA at room temperature for 45 min in dark) prior to each of process 1, 2, and 3. Denaturation and reduction opens the three-dimensional glycoprotein structure, thus permitting an enzyme accessing the glycosylation and/or proteolytic sites. In process 1, step 1, the denatured and reduced glycoprotein sample is subjected to deglycosylation using PNGase F to generate detached N-linked glycans and an intact protein. In step 2, the detached N-linked glycans then are isolated from the intact protein using 10,000 Da molecular weight cut off filter to produce a glycan fraction (Fraction A) and a protein fraction (Fraction B). The glycan fraction (Fraction A) is collected for glycan analysis by LC-MS/MS using CID-MS² and CID-MS³.

Process 2 involves the use of multiple enzymes including PNGase F (see step 1) to generate a mixture of non-glycosylated and deglycosylated peptides (Fraction C). The intact protein fractionation (Fraction B) obtained in Process 1 (Step 2) can also be used to generate fractionation (Fraction C) by adding the same multiple enzymes without PNGase F as described in Process 2 (Step 1). The resulting fraction (Fraction C) then is subjected to LC-MS/MS analysis (Step 2) using CID-MS², CID-MS³, and/or HCD-MS² (FIG. 6, Process 2/Step 3).

Process 3 produces non-glycosylated and glycosylated peptides (Fraction D) using the same multiple enzyme(s) as process 2 except no PNGase F (step 1). ETD can preserve labile post-translational modifications (PTMs) so as to preserve glycans attached on a peptide backbone. Alternatively, CID and/or EICD can generate fragment ions predominantly from cleavage of glycosidic bonds on glycans without breaking the peptide amide bonds (Wu et al. (2007) supra; Ye et al., ANAL. CHEM. (2013) 85(3): 1531-1539; Miller et al., J. PHARM. SCI. (2011) 100(7): 2543-2550). Hence, glycosylation sites can be identified using CID, HCD and ETD. For example, glycosylated (N-linked) and nonglycosylated peptides (Fraction D) are subjected to LC-MS/MS analysis. ETD (ETD-MS²), CRCID (CID-MS³), CID (CID-MS²) and/or HCD (HCD-MS²) are used to fragment glycosylated peptides to determine N-glycosylation sites (FIG. 6, Process 3/Step 2). CID (CID-MS²/-MS³) and/or HCD (HCD-MS²/-MSⁿ) are applied to glycosylated peptides, which enable determining site-specific glycans (Process 3/Step 2). Matching observed glycopeptide masses with theoretical masses of glycans and peptides indicate if the protein is properly glycosylated.

Nonglycosylated peptides present in Fraction D can be distinguished from glycosylated peptides once N-linked peptides are identified. The sequence of nonglycosylated peptides can be obtained using CID-MS² and/or HCD-MS² (Process 3/Step 6). Through mapping of deglycosylated peptides (Process 2/Step 2), nonglycosylated peptides (Process 2/Step 2 and Process 3/Step 3), and glycosylated peptides (Process 3/Step 2), the N-linked glycosylation sites can be determined.

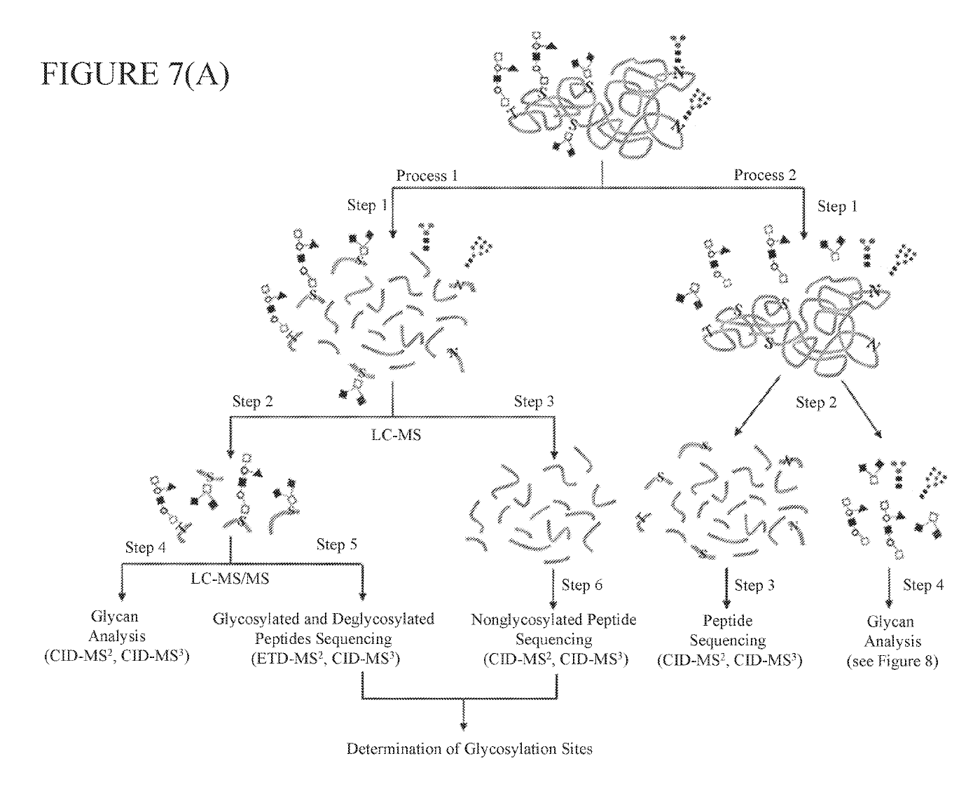

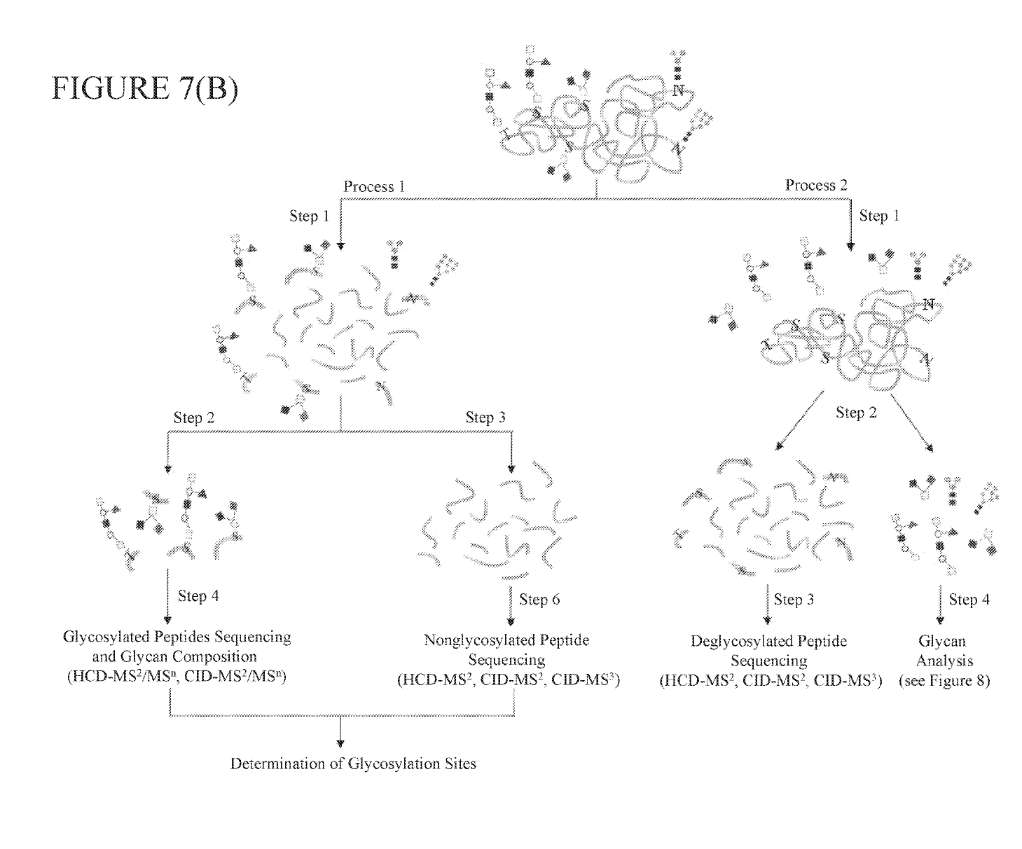

The characterization of O-linked glycosylated biologics is more complicated because there is no single enzyme available to cleave an “intact” O-linked glycan complex from serine or threonine residues on the protein. Traditionally, O-glycans are released through β-elimination using strong bases such as sodium hydroxide or hydrazinolysis using hydrazine (Patel et al., BIOCHEMISTRY (1993) :32: 679-693). Although, β-elimination reaction can remove an intact O-glycan, the protein can be degraded under alkaline conditions. In addition, O-linked glycoproteins may contain N-linked glycans. Therefore, as shown in FIG. 7(A) and FIG. 7(B), the characterization method for N-linked glycosylation can be modified to enable O-linked analysis, and involves two processes: an enzymatic process (Process 1) and a chemical and enzymatic process (Process 2).

As shown in FIG. 7(A), the glycoprotein sample is subjected to denaturation, reduction, and alkylation prior to Process 1 and Process 2. In Process I (Step 1), the reduced glycoprotein is digested with multiple enzymes including PNGase F to generate O-linked glycopeptides, deglycosylated and non-glycosylated peptides. Then the digest sample is subjected to LC-MS analysis. Using the predicted masses of known and possible O-linked glycopeptides, they can be measured under a selected ion monitoring (SIM) mode on a mass spectrometer (Step 2). Once detecting the masses matched with predicted O-linked glycopeptides, O-linked glycans (Step 4) can be fragmented using CID (CID-MS²) and/or FWD (HCD-MS²) to determine site-specific O-glycans. O-glycopeptides are sequenced using ETD (ETD-MS²), CRCID (CID-MS³), (CID-MS²), or HCD (HCD-MS²) (Process 1/Step 5). Non-glycosylated peptides can be located on the LC-MS chromatogram based on their predicted masses (Step 3). Non-glycosylated peptide sequencing is carried out using CID (CID-MS²) and HCD (HCD-MS²) (Step 6). Through mapping of glycosylated and non-glycosylated peptides (Step 5 and Step 6)), O-linked glycosylation sites are determined. As shown in FIG. 7(B), steps 4 and 5 can be combined, and glycosylated peptide sequencing and glycan analysis can be performed in a single step using HCD (HICD-MS²/-MSⁿ) and CID (CID-MS²/-MSⁿ).

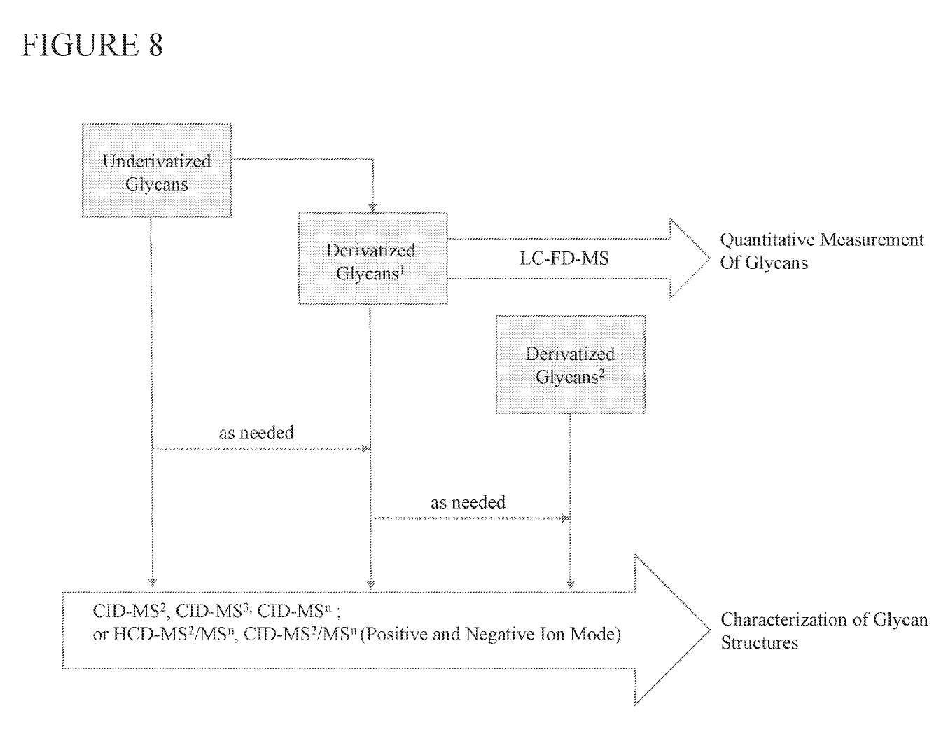

Process 2 of FIG. 7(A) can be used for glycan analysis. Chemicals (for example, GlycoProfile™ β-Elimination Kit, Sigma) can be used to free glycans (both O- and N-linked glycans) (Step 1) from the glycoprotein. The released glycans then are separated from an intact protein using 10,000 Da molecular weight cut off filter (Step 2). The resulting, harvested glycans are subsequently subjected to glycan analysis (Step 3). The analysis of the glycans can be important in the characterization of the biologic because the heterogeneity of glycoprotein is mainly due to glycan content, which can differ by sequence, chain length, branching site, and position of linkage to the peptide chain. For example, there are four different N-linkages to asparagine (Asn) residues on N-linked glycoproteins: 1) N-acetylglucosamine (GlcNAc); 2) N-acetylgalactose (GaINAc); 3) glucose; 4) rhamnose. N-glycans are usually attached to a protein at Asn-X-Ser or Asn-X-Thr sequences, where X can be any amino acid except Pro. The most common N-linkage is GlcNAc-Asn, consisting of three general types of N-gly cans: oligomannose, hybrid, and complex (Kornfeld et al., ANN. REV. BIOCHEM. (1985) 54: 631-664). Importantly, glycan analysis is always included as part of quality control for glycoprotein drug products since sugar moieties involve in protein folding, stability, and biological functions. Glycan analysis can provide qualitative (i.e., characterization of glycan structures, such as glycan type including sequence, chain length, position of linkage) and quantitative (for example, relative amounts of each glycan type) measurement see, FIG. 8).

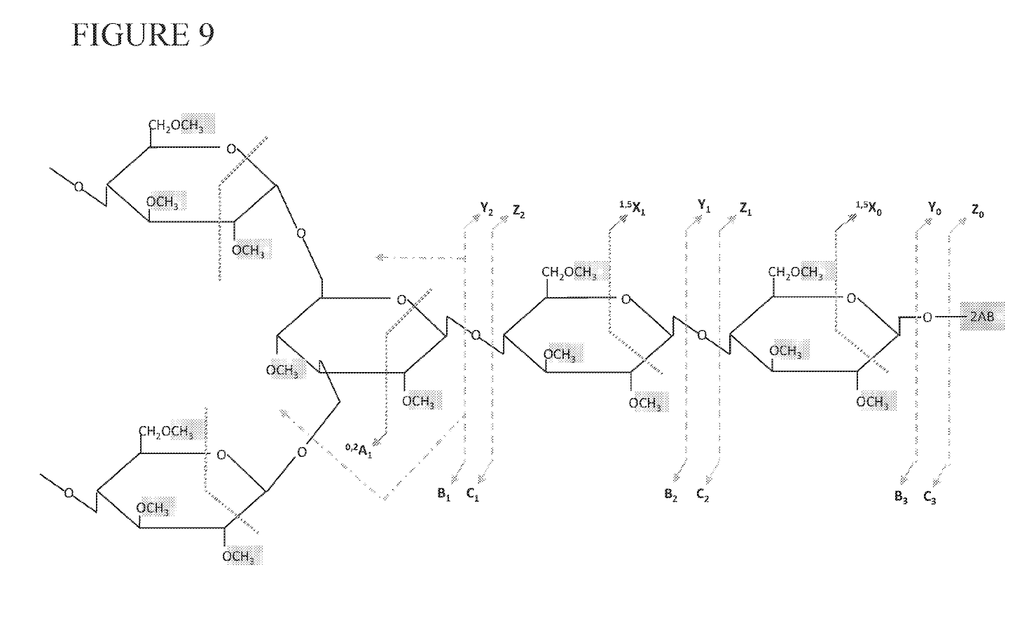

Analysis of the glycan moieties attached to a protein can be performed through enzymatic or chemical processes (referred to deglycosylation) as illustrated in FIG. 6 (Process 1, Step 1) arid FIG. 7(A) and (B) (Process 2, Step 1). For example, following the characterization method for N-linked glycosylated proteins (FIG. 6, Process Step 1→Step 2→Step 3), the released N-glycan sample is analyzed by LC-MS using a HILIC LC column (for example, Thermo GlycanPac AXH, 1.9 μm, 2.1 mm id×15 cm) through a linear gradient from high to less organic phases (for example, from 80/20 to 60/40 acetonitrile/water in 40 minutes) at a flow rate of 200 μL/min. Each type of glycan molecule is quantitatively measured under a selected ion monitoring mode (SIM) on a mass spectrometer according to their predicted masses. CID (CID-MS²) fragmentation then is used to fragment each selected glycan ion, thus permitting to assign the glycan structure. For a comparability study (for example, two different lots of biologic drugs are produced in manufacture, Lot 1 vs. Lot 2), the released glycans from Lot 1 and Lot 2 are profiled by LC-MS. Through LC-MS profiling, most glycan species can be distinguished between Lot 1 and Lot 2 based on the LC separation and their masses. If glycan isomers cannot be separated by LC; the MS/MS experiments using CID-MS² and/or CID-MS³ can further break their molecules to produce unique fragmentation ions (product ions). Use of a combination of theoretic masses and fragmentation spectra to search against a glycan database (for example, a glycan library built using Glycoworkbench) can assign glycan identities. Nevertheless, some of the glycans may be difficult to measure quantitatively because of poor ionization of glycan structures by MS, or identify because of the complex isomeric nature of glycans. Labeling glycans via derivatization can be used to improve the detection of glycans (Ruhaak et al., ANAL. BIOANAL. CHEM. (2010) 397: 3457-3481). Depending upon the complexity of glycan structures, a multi-tier strategy can be applied for quantitative and qualitative glycan analyses (see, FIG. 8). For example, released glycoforms are labeled with fluorescent dye such as 2-aminobenzamide (2-AB) to create derivatized glycans (denoted as Derivatized Gly cans¹, FIG. 8) through reductive amination. Then, 2-AB labeled glucans are quantitatively analyzed by LC-FD-MS. In order to assign glycan structures, a multi-stage approach is used as illustrated in FIG. 8. For example, LC-MS/MS using CID (CID-MS², CID-MS³, and possibly CID-MSⁿ) and/or HCD (HCD-MS²) are used to characterize underivatized glycans as the first-stage. Based on the glycans of interests, some glycans may need to be derivatized. For example, the reducing end of branching glycans can be labeled for example, labeled with 2-AB and then permethylated to provide another set of derivatized glycans (denoted as Derivatized Glycans², FIG. 8). Permethylation of glycans replaces hydrogens on hydroxyl, amine, and carboxyl groups with methyl groups. Then CID-MS² or HCD-MS² can be used to break glycosidic bonds to generate fragment B, C, Y, Z ions (FIG. 9, dashed lines) and to cleave cross-ring bonds to produce fragment A and X ions (FIG. 9, dotted lines) (Domon et al., GLYCOCONJUGATE JOURNAL, (1988) 5: 397-409). Generally, permethylated glycans are analyzed by MS under a positive ionization mode. By monitoring predicted masses of permethylated glycans as precursor ions, methylated glycans are subsequently fragmented by CID (CID-MS², CID-MS³, and possibly MSⁿ). Because methylated functional groups (for example, hydroxyl, amine, and/or carboxyl groups) noted on glycans cannot be the linkage sites, this permits the assignment of branching linkage position(s). Additionally, D ions can be generated by B and Y cleavages of two glycosidic bonds adjacent to the monosaccharide unit at the branch (FIG. 9, dash-dot lines) under a negative ionization mode by MS (Harvey, MASS SPCETROMETRY REVIEWS (1999) 18: 349-451). The use of fragmentation patterns (for example, A, B, C, X, Y, Z, and D ions) and derivatization method(s) (for example, 2-AB labeling or permethylation) can permit the assignment of branching glycan structures.

### IV. Creation of the In Vivo Comparability Profile

Once administered, extracted and analyzed using the method described above, the resulting data may be used to create an in vivo comparability profile which includes comparative data indicative of the behavior of the candidate and reference biologic drugs in vivo following administration. The candidate and reference biologics may be extracted at two or more different time points, and from two or more different physiological compartments, and the resulting data incorporated in to the comparability profile.

A. Determination of the Effect of In Vivo Residence on the Structure and Function of the Biologic

Any alterations in the structural features and optional function characteristics of the candidate and reference molecules can be determined and resulting information incorporated in the in vivo comparability profile.

1. Structure

The structural features of the candidate biologic drug and the reference biologic can be analyzed following in vivo residence using the approaches described above in Section III. The resulting data can identify those structures or substructures within the candidate and reference biologic susceptible to modification or degradation.

By way of example, the candidate and reference biologic is administered to a subject and extracted after one or more predetermined time periods from one or more physiological compartments of the subject, for example, blood, lymph, muscle tissue, fatty tissue. After the predetermined period of time, the structure of the candidate or reference biologic or metabolites thereof are interrogated by one or more of the analytic techniques discussed above. The method can be repeated using one or more different time periods, or using samples extracted from one or more of the other physiological compartments. Any differences (or the converse, no differences) to the structure of the candidate and reference biologics following in vivo residence can be assimilated in the comparability profile.

In addition, biologics can also break down into fragments as they are metabolized or otherwise broken down in vivo. For example, a protein with quaternary structure can create peptide fragments by mechanisms other than by enzymatic or chemical digestion. Rather, stress induced fragmentation can occur by hydrolysis of amide bonds in peptide backbones. For example, Asp-Gly and Asp-Pro peptide bonds are particularly susceptible to hydrolysis. The stress induced fragments can be determined using, for example, bel electrophoresis together with LC-MS/MS analysis. By way of example, two samples are created: a control sample, and a sample administered to a subject and extracted. To detect fragmentation in the administered sample, control and administered samples are subjected to gel electrophoresis under non-reducing conditions. If fragmentation occurs during in vivo residence, lower molecular weight fragments are observed following gel electrophoresis which are not observed in the control sample. The gel pieces corresponding to lower molecular weight fragments in the administered sample and the intact protein in the control sample are excised from the gel. Following in-gel digestion with enzyme(s), the fragmented protein degradation products are identified by LC-MS/MS using CID and/or ETD.

Collectively, the information resulting from the foregoing analyses provides insights into the structural or the substructural features of the candidate and reference biologic that are affected during in vivo residence. The resulting structural information can also be correlated with biological function, if and/or when biological data becomes available, to determine how administration to a subject affects the safety, potency and/or efficacy of the reference biologic.

2. Function

In addition to analyzing the effect of in vivo residence on structure of the biologic, it can be helpful to determine if the structural changes result in a measureable effect on safety, efficacy and potency. The structural changes may vary from having little or no effect on function to having profound effect on function. As a result, the biological information can further define those aspects of the structure of the candidate and reference biologics that are critical to safety, purity, and/or potency.

The effect of in vivo residence can be determined by analyzing the extracted biologics or metabolites via one or more biological assays used to measure biological function. For example, the activity, in particular the pharmacological activity, of the biologic can be evaluated by in vitro and/or in vivo functional assays. The assays may include, but are not limited to, bioassays, biological assays, binding assays, and enzyme kinetic assays. The particular assays, however, will depend upon the actual biologic being tested. For example, in the case of antibodies, the ability to bind to a target ligand, as well as the binding affinity are important biological assays. Those assays can be used with the extracted molecules to determine what effect, if any, in vivo residence on the biological activity of the protein.

Potency is another important criteria, and represents a measure of relevant biologic function coupled to therapeutic activity of the drug products. Potency tests can be performed to assure structural attributes associated with a product (see “Guidance for Industry, Potency Tests for Cellular and Gene Therapy Products” U.S. Department of Health and Human Services Food and Drug Administration (2011)). After confirming the structural changes that result from in vivo residence, potency testing using an appropriate bioassay can be carried out to measure the therapeutic activity (i.e., strength) for the extracted samples. If no difference is observed, then it would appear that a particular structural modification does not have a substantial effect on efficacy.

3. Creation of the Comparability Profile

Once the structural and the optional function features of the candidate and reference biologics have been determined, the resulting data can be analyzed to determine the effect of in vivo residence on the biologic. The data preferably is analyzed computationally using a conventional computer or computer system to identify structural changes, and to identify critical structure or substructures of the biologic.

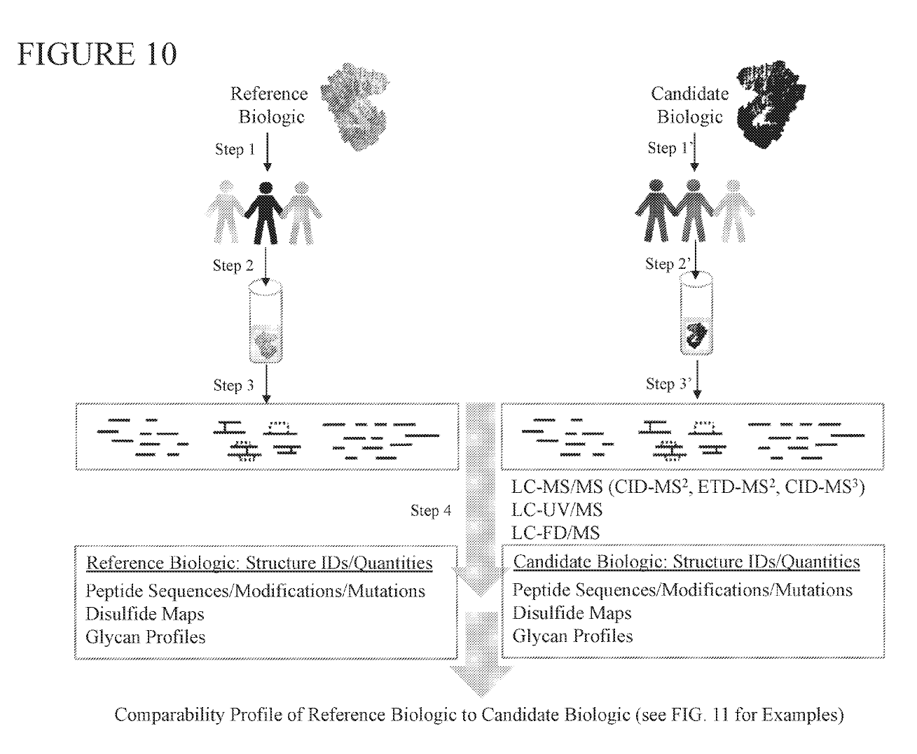

This information can then be assimilated to produce a comparative chart (see, e.g., FIGS. 10, 11, 12 and 14) that identifies and may quantitatively or statistically compare one or more structural features, or groups of structural features, of the candidate and reference biologics that are modified in vivo, which may affect the function of the biologic, and/or which may have limited or no effect on the function of the biologic. The in vivo comparability profile may include data indicative of whether a feature is more or less common in the candidate biologic drug or a metabolite thereof, relative to the reference biologic drug or a corresponding metabolite thereof. For example, the profile may indicate that the candidate biologic drug is phosphorylated at a different amino acid residue than in the reference biologic drug, or it may indicate that the candidate biologic drug is phosphorylated more or less often at a particular residue than in the reference biologic drug. Similarly, structural similarities between the candidate biologic or a metabolite thereof and a reference biological or a corresponding metabolite thereof can be used to support the comparability and similarity of the candidate biologic and the reference biologic.

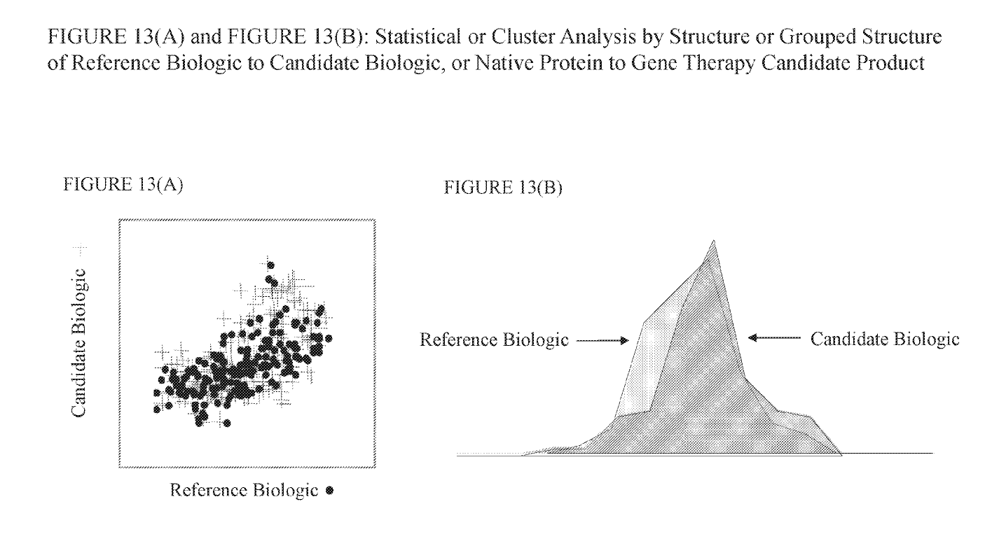

The data analysis and the creation of the in vivo comparability profile can be conducted using commercially available software packages, including for example, SAS v.9.4 (SAS Institute Inc.). or the Spotfire Platform (TIBCO Spotfire). An exemplary cluster analysis and peak profile analysis of in vivo comparability data are depicted in FIGS. 13(A) and 13(B) respectively.

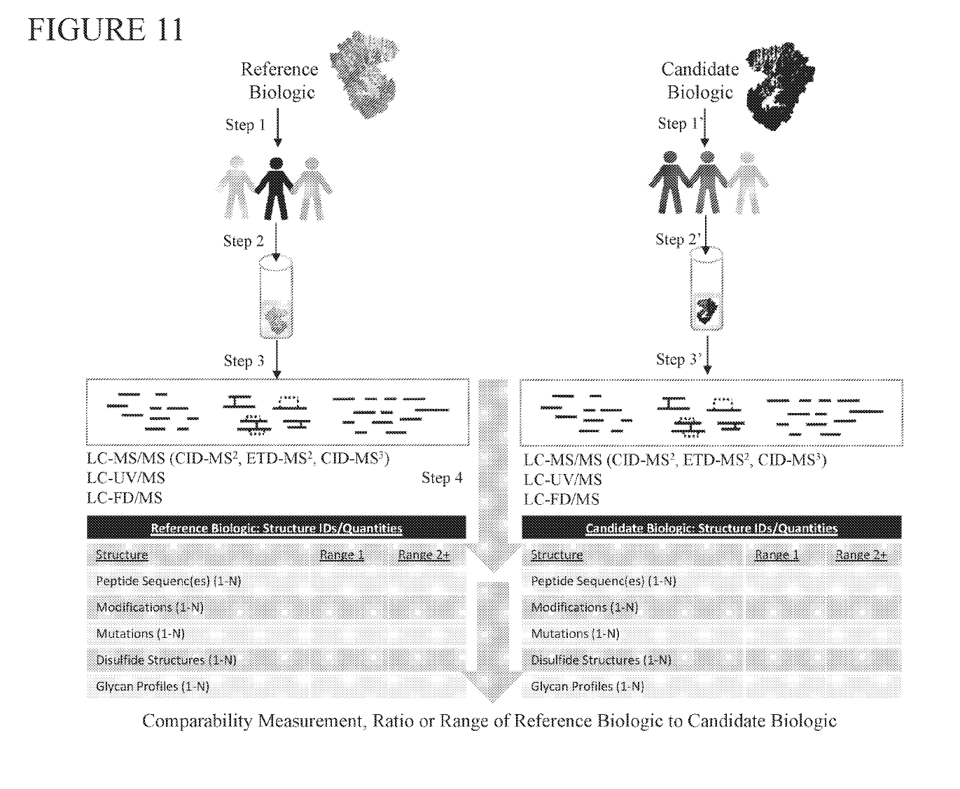

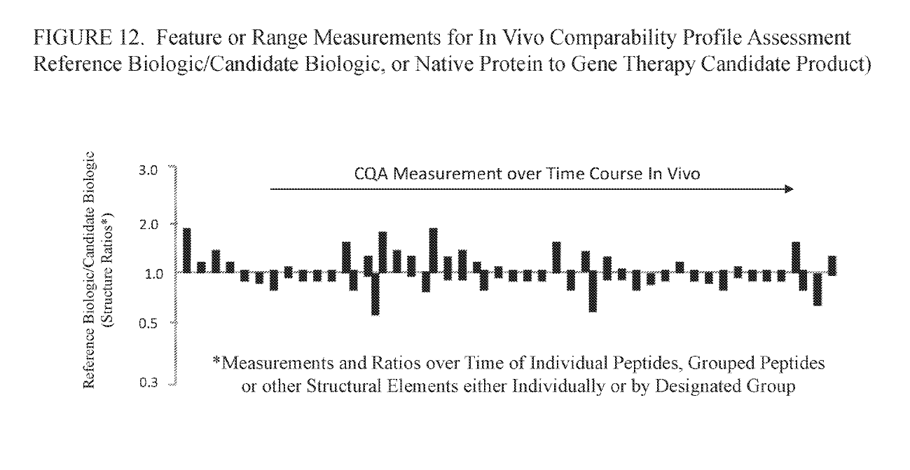

By way of example, FIG. 10 depicts an exemplary process for generating an in vivo comparability profile. In Step 1 and 1′, a reference biologic or candidate biologic is administered to a subject, in Step 2 and 2′, the biologic and optionally one or more of its metabolites are extracted from the subject. In Step 3 and 3′, the biologic and optionally one or more of its metabolite are fragmented, and in Step 4, the resulting fragments are subject to mass spectroscopic analyses. Categories of the analyses' resulting output are provided. It is understood that, in FIG. 10, each of Steps 1, 2, 3 and 4 can be performed before, contemporaneous with, or after corresponding Steps 1′, 2′, 3′, and 4′. FIG. 11 depicts a schematic representation of the exemplary process depicted in FIG. 110 further depicting exemplary mass spectroscopic analysis output data. FIG. 12 depicts an exemplary feature or range measurement for in vivo comparability profile assessment, showing changes in the ratio of relative abundance of a feature or group of features over time between a candidate and reference biologic. FIG. 13 depicts an exemplary cluster analysis (FIG. 13(A)) and an exemplary peak analysis (FIG. 13(B)), showing the results of statistical analysis of mass spectroscopic data from reference and candidate biologics, and which may be used to demonstrate in vivo comparability between the reference and candidate biologics

The resulting in vivo comparability profile can also be used as a standard in the development of an innovator biologic drug or a biosimilar. In the case of a biosimilar, the in vivo comparability profile can be used to support the determination that a candidate biologic is biosimilar or is highly similar to a reference biologic. For example, the profile may be used (a) as a target to design the biosimilar development and regulatory processes, and (b) as a qualification of biosimilarity for a given protein or batch or proteins as related to a previously marketed protein approved by a regulatory agency. The approach can also be used to determine whether another biologic, for example, made under different conditions (for example, expressed using a different expression vector, expression host, or culture conditions) is substantially similar to the reference biologic. The method may comprise analyzing a batch of material to assure that its in vivo performance attributes satisfy certain features identified in the in vivo comparability profile. Once satisfied, ⁻the batch of material can then be marketed as an approved drug.

Furthermore, the in vivo comparability profile can be used to facilitate the development of gene therapy-based approaches to replace, augment or repair an endogenous protein. For example, the expression product of a transfected gene therapy construct can be affinity purified, modified, as appropriate, and analyzed to produce a characterization profile that is compared to the endogenous molecule (for example, endogenous protein target of the gene therapy augmentation or modulation) where the resulting in vivo comparability profile shows that the expression product has the relevant or the similar structural features optional functional attributes) as the endogenous target.

In one embodiment, the expressed gene product can be extracted from cells transfected with a gene for a protein of interest, for example, a growth hormone, isolated, fully characterized, and compared to the corresponding recombinant human protein, for example, a growth hormone drug and/or to the corresponding endogenous growth hormone to develop a comparability profile with regards to the critical or target structural features and functional attributes. In another embodiment, the expression product from cells transfected with a gene encoding a protein of interest, for example, α-galactosidase A, can be compared to the corresponding recombinant protein drug, for example, agalsidase β or agalsidase α, and/or to the corresponding purified native α-galactosidase A enzyme produced in normal human tissue (for example, kidney). These key structural and functional attributes can be used to demonstrate comparability or similarity of the gene therapy expression product, recombinant product, and endogenous product.

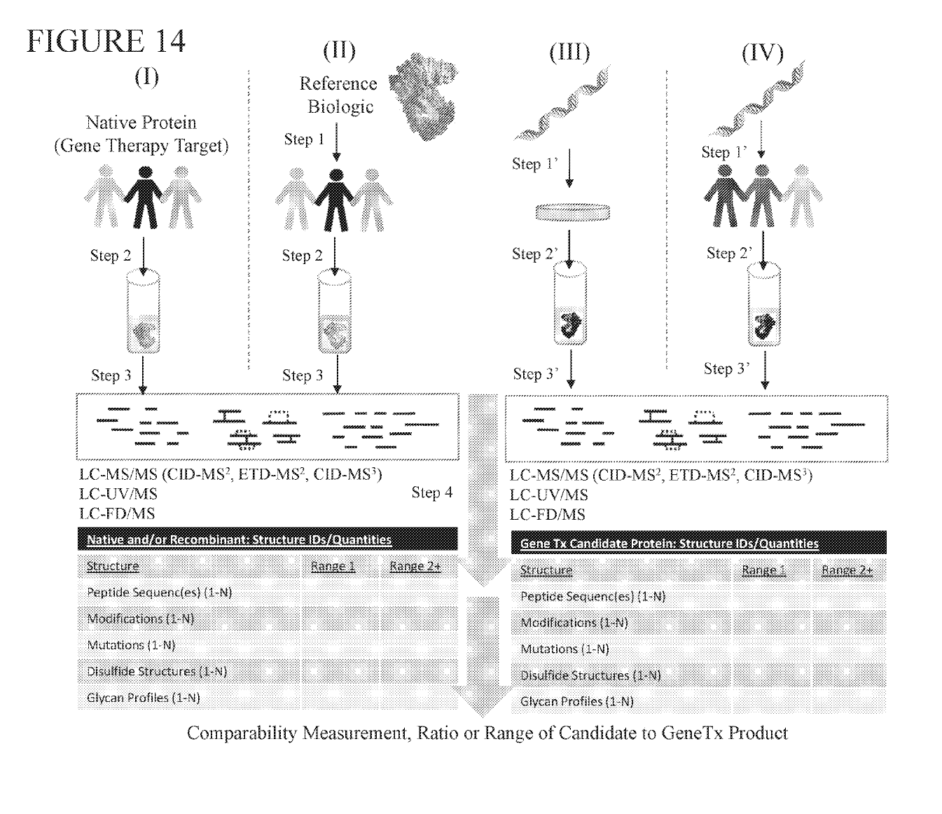

FIG. 14 depicts two exemplary processes for generating in vivo comparability profiles comparing either a native protein (I) or reference biologic (II) to the expression product of a gene therapy construct that is expressed in cell culture (III), or in a subject (IV). In Step 1 the reference biologic is administered to the subject, and in step 2, the reference biologic and optionally its metabolites are extracted. As shown in (I), if the reference is the endogenous protein, then the administration step (Step 1) is omitted, and the protein is extracted in Step 2. In Step 1′, the gene therapy construct is added to cells in culture (III) or administered to a subject (IV), and in Step 2′, the resulting expression product is extracted, In Steps 3 and 3′ the extracted biologics are fragmented, and in Step 4, the fragments are subject to mass spectroscopic analyses. An exemplary output of the mass spectroscopic data analyses is depicted.

### INCORPORATION BY REFERENCE

The entire disclosure or each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of assessing in vivo comparability of a candidate biologic drug to a reference biologic drug, the method comprising the steps of:
(a) generating through mass spectroscopic analysis data indicative of the structure of the candidate biologic drug or a metabolite thereof following extraction from a sample removed from a subject at a first time interval following administration of the candidate biologic drug to the subject; and
(b) comparing the data generated during step (a) to mass spectroscopic analysis data indicative of the structure of the reference biologic drug or a metabolite thereof generated through analysis of a sample taken at the same time interval from a subject to whom the reference biologic drug had been administered, thereby to produce an in vivo comparability profile of the candidate biologic drug to the reference biologic drug.

2. The method of claim 1 comprising the additional step of using the comparability profile to determine the metabolic comparability of the candidate biologic drug to the reference biologic drug.

3. The method of claim 1 or 2, wherein the comparability profile contains data indicative of the structural or metabolic profile of the reference biologic drug and the structural or metabolic profile of the candidate biologic drug over time following administration to the subject.

4. The method of any one of claims 1-3, wherein the comparability profile comprises one or more sets of data indicative of:
(a) a structural feature that is the same or different when comparing the candidate biologic drug or a metabolite thereof and the reference biologic drug or a corresponding metabolite thereof; and optionally
(b) data indicative of whether differences in a structural feature between the candidate biologic drug and the reference biologic drug substantially affect a biological activity of the candidate biologic drug.

5. The method of any one claims 1-4, wherein the sample is a cell, tissue or body fluid sample.

6. The method of any one of claims 1-5, further comprising repeating step (a) at two or more time intervals following administration so as to produce a comparability profile of the candidate biologic drug relative to the reference biologic drug as a function of time in vivo.

7. The method of any one of claims 1-6, wherein the subject in step (a) is a human, and/or the subject in step (b) is a human, and optionally wherein the subject in step (a) and the subject in step (b) are the same subject.

8. The method of any one of claims 1-6, wherein the subject in step (a) is an animal, and/or the subject in step (b) is an animal, and optionally wherein the subject in step (a) and the subject in step (b) are the same subject.

9. The method of any one of claims 1-8, wherein the mass spectrometry analysis data is generated by fragmenting or chemically modifying the reference biologic drug, the candidate biologic drug, or metabolites thereof before or while subjecting the sample to mass spectrometric analysis.

10. The method of any one of claims 1-9, wherein the generation of mass spectrometry data is effected through one or more techniques selected from the group consisting of electron transfer dissociation mass spectrometry, collision induced dissociation mass spectrometry, higher-energy collisional dissociation mass spectrometry, electron capture dissociation mass spectrometry, infrared multi-photon dissociation mass spectrometry, ultraviolet photodissociation mass spectrometry, hydrogen/deuterium exchange mass spectrometry, MS², MS^{'}, and LC-MS.

11. The method of any one of claims 1-10, wherein mass spectrometry analysis data are generated by one or more analytical techniques selected from the group consisting of fluorescent spectra, light scattering spectroscopy, electrophoresis, selective proteolysis, UV spectra analysis, IR spectra analysis, Hydrogen-Deuterium exchange analysis, and MRI spectra analysis.

12. The method of any one of claims 1-11, wherein the comparability profile generated in step (b) is of sufficient detail to permit qualification of the candidate biologic drug as biosimilar to the reference biologic drug.

13. The method of any one of claims 1-11, wherein the comparability profile generated in step (b) is of sufficient detail to permit qualification of the candidate biologic drug as substantially the same as the reference biologic drug.

14. The method of any one of claims 1-11, wherein the candidate biologic drug and the reference biologic correspond to different manufacturing lots of the same biologic drug, and the comparability profile generated in step (b) is of sufficient detail to serve as criteria to qualify the release of the manufacturing lot containing the candidate biologic drug.

15. The method of any one of claims 1-14, wherein the candidate biologic drug is an expression product produced by expression of a gene during gene therapy and the reference biologic drug is a native expression product whose level is intended to be modulated by the gene therapy.

16. The method of any one of claims 1-14, wherein the candidate biologic drug and the reference biologic drug are a gene therapy delivery construct.

17. The method of any one of claims 1-15, wherein the biologic drug is a protein or peptide.

18. A method of assessing in vivo comparability of a candidate biologic drug to a reference biologic drug, wherein the candidate biologic drug is a protein produced by expression of a gene during gene therapy that corresponds to a native protein whose level is intended to be modulated by the gene therapy and the reference biologic drug is a recombinant protein produced in a cell-line that corresponds to the native protein, the method comprising the steps of:
(a) generating through mass spectroscopic analysis data indicative of the structure of the candidate biologic drug or a metabolite thereof following extraction from a sample removed from a subject at a first time interval following administration of the candidate biologic drug to the subject; and
(b) comparing the data generated during step (a) to mass spectroscopic analysis data indicative of the structure of the reference biologic drug, thereby to produce an in vivo comparability profile of the candidate biologic drug to the reference biologic drug.

19. The method of claim 18, wherein the comparability profile comprises one or more sets of data indicative of:
(a) a structural feature that is the same or different when comparing the candidate biologic drug or a metabolite thereof and the reference biologic drug; and optionally
(b) data indicative of whether differences in a structural feature between the candidate biologic drug and the reference biologic drug substantially affect a biological activity of the candidate biologic drug.

20. The method of claim 18 or 19, wherein the sample is a cell, tissue or body fluid sample.

21. The method of any one of claims 18-20, further comprising repeating step (a) at two or more time intervals following administration so as to produce a comparability profile of the candidate biologic drug relative to the reference biologic drug as a function of time in vivo.

22. The method of any one of claims 18-21, wherein the subject in step (a) is a human.

23. The method of any one of claims 18-21, wherein the subject in step (a) is an animal.

24. The method of any one of claims 18-23, wherein the mass spectrometry analysis data is generated by fragmenting or chemically modifying the reference biologic drug or metabolites thereof or the candidate biologic drug before or while subjecting the sample to mass spectrometric analysis.

25. The method of any one of claims 18-24, wherein the generation of mass spectrometry data is effected through one or more techniques selected from the group consisting of electron transfer dissociation mass spectrometry, collision induced dissociation mass spectrometry, higher-energy collisional dissociation mass spectrometry, electron capture dissociation mass spectrometry, infrared multi-photon dissociation mass spectrometry, ultraviolet photodissociation mass spectrometry, hydrogen/deuterium exchange mass spectrometry, MS², MS³and LC-MS.

26. The method of any one of claims 18-26, wherein mass spectrometry analysis data are generated by one or more analytical techniques selected from the group consisting of fluorescent spectra, light scattering spectroscopy, electrophoresis, selective proteolysis, UV spectra analysis, IR spectra analysis, Hydrogen-Deuterium exchange analysis, and MRI spectra analysis.

27. The method of any one of claims 18-26, wherein the comparability profile generated in step (b) is of sufficient detail to permit qualification of the candidate biologic drug as substantially the same as the reference biologic drug.

28. The method of any one of claims 1-27, wherein, in step (a), the data indicative of the structure of the candidate molecule or the metabolite thereof comprises a normalized measure of a modified form of a structural attribute corresponding to the amount of a modified form of a structural attribute relative to the total amount of the structural attribute (a sum of modified attribute and unmodified attribute) in the sample being tested.
